(19) United States
(12) Patent Application Publication
(10) Pub. No.: US 2007/0248546 A1
(43) Pub. Date: Oct. 25, 2007

(54) Clinical algorithm for excluding patients identified in virtual imaging

(75) Inventor: Anthony P. Shuber, Mendon, MA (US)

Correspondence Address: WOLF GREENFIELD & SACKS, P.C., 600 ATLANTIC AVENUE, BOSTON, MA, 02210-2206, US

(73) Assignee: Exact Sciences Corporation, Marlborough, MA (US)

(21) Appl. No.: 11/595,593

(22) Filed: Nov. 9, 2006

Related U.S. Application Data
(60) Provisional application No. 60/735,979, filed on Nov. 9, 2005.

(51) Int. Cl. A61K 49/04

(52) U.S. Cl. 424/9.4

(57) ABSTRACT

Aspects of the invention relate to clinical triage protocols for screening a patient population using virtual imaging techniques, for example, virtual colonoscopy. Methods for increasing the specificity of a virtual imaging procedure are provided. In aspects of the invention, colonic effluents are analyzed using one or more molecular detection assays.

### RELATED APPLICATION

This application claims priority under 35 U.S.C. §119(e) from U.S. provisional application Ser. No. 60/735,979, filed Nov. 9, 2005, the content of which is incorporated herein in its entirety.

### FIELD OF THE INVENTION

The invention relates generally to clinical algorithms for analyzing patients using virtual imaging.

### BACKGROUND OF THE INVENTION

Virtual colonoscopy using imaging devices are used to identify patients for invasive testing.

### SUMMARY OF THE INVENTION

In one aspect, the invention relates to methods and composition for increasing the specificity of a virtual colonoscopy. A virtual colonoscopy can detect many different colonic lesions and typically cannot distinguish between many cancerous and non-cancerous lesions. Accordingly, virtual colonoscopy is a low specificity technique that detects many false positives when used as a screen to detect cancerous or pre-cancerous colonic lesions in subjects. In general, a patient that is identified as positive by virtual colonoscopy is subsequently tested using an invasive colonoscopy and/or tissue biopsy. According to the invention, invasive testing of false positive patients imposes a cost and time burden on health care organizations and involves unnecessary risk and discomfort for the patients. In one aspect, the invention provides a method for increasing the specificity of a virtual colonoscopy by providing an adjunct molecular test that can be performed on colonic effluent generated as part of the virtual colonoscopy analysis. According to aspects of the invention, a high sensitivity molecular analysis on colonic effluent can be used to exclude false positives from subsequent invasive testing. However, patients who are identified as positive in a virtual colonoscopy and in an adjunct molecular assay may be subsequently evaluated using one or more invasive techniques (e.g., to identify or detect cancerous or precancerous lesions).

In aspects of the invention, a patient may be a mammal, e.g. a human, a cat, a dog, a monkey or any other mammal.

Aspects of the invention provide a clinical algorithm that acts as a triage for virtual colonoscopy. In one aspect, colonic effluent is collected from patients being screened via virtual colonoscopy. In one embodiment, a colonic effluent sample from each patient is processed and analyzed during the colonoscopy procedure. In one embodiment, a colonic effluent analysis is performed only for those patients that are identified as positive (or questionable) in the virtual colonoscopy. The analysis may be performed on colonic effluent obtained at any stage during the virtual colonoscopy. In one embodiment, the colonic effluent may be collected during insufflation of the colon. However, it should be appreciated that colonic lavage effluent obtained prior to the virtual colonoscopy may be used. It also should be appreciated that colonic effluent recovered at the end (or immediately after) the virtual colonoscopy may be used. The amount of colonic effluent required for analysis will depend on the concentration of molecular markers (e.g., cells, cell free nucleic acid, cellular debris or any combination thereof) in the effluent. In some embodiments, the effluent may be concentrated (e.g., in a dehydration step) in an initial step prior to processing for subsequent analysis. In some embodiments, colonic effluent obtained at different stages (e.g., any combination of two or more of the following effluents: effluent retrieved during lavage of the colon in preparation for virtual colonoscopy, effluent retrieved during the virtual colonoscopy imaging step, effluent retrieved during insufflation, effluent retrieved after the imaging process is ended, effluent retrieved after insufflation is ended, etc.) may be analyzed. Effluent from different stages may be combined. Alternatively, effluent from different stages may be separately analyzed and the results compared for consistency or statistical significance.

In one aspect, colonic effluent is stabilized for subsequent analysis. In some embodiments, stabilization buffer is added to colonic effluent after retrieval. In some embodiments, colonic effluent is retrieved into a container that already contains stabilization buffer. In some embodiments, a chelator of divalent cations (e.g., EDTA) is added to colonic effluent (e.g., as a dry powder or liquid) after retrieval. In some embodiments, colonic effluent is retrieved into a container that already contains (e.g., as a dry powder or liquid) a chelator of divalent cations (e.g., EDTA). In some embodiments, the colonic effluent may itself contain stabilization buffer. For example, a stabilization buffer may be added to any solution that is introduced into the colon during the preparative lavage stage and/or during insufflation and/or during virtual imaging and/or at the end of (e.g., immediately after) the virtual imaging stage. Accordingly, one aspect of the invention relates to washing a patient's colon with a stabilization buffer (e.g., a buffer containing a chelator such as EDTA).

It also should be appreciated that aspects of the invention may include analyzing stool samples. In some embodiments, a patient may provide a stool sample that is obtained prior to preparation for the virtual colonoscopy. This sample may analyzed during the virtual colonoscopy for all patients. Alternatively, this sample may be analyzed only for those patients that are identified as positive (or suspect) during the virtual colonoscopy. In some embodiments, a patient may provide a stool sample that is obtained after the virtual colonoscopy. In some embodiments, all patients provide a stool sample after the virtual colonoscopy. In other embodiments, only those patients that are identified as positive (or suspect) during the virtual colonoscopy are asked to provide a stool sample after the virtual colonoscopy. In some embodiments, one or more stool samples may be analyzed in addition to one or more colonic effluent samples. However, in some embodiments, a stool sample may be used instead of colonic effluent and the results from the stool sample analysis alone may be used as an adjunct to the virtual colonoscopy.

It should be appreciated that any single molecular analysis (e.g., of colonic effluent or stool sample) may be sufficient to exclude (or include) a patient for subsequent invasive analysis. However, in some embodiments, an analysis of several samples may be used. In some embodiments, the results obtained from the molecular analysis may be combined with other factors (e.g., age, disease history, gender, fecal occult blood test results, physical characteristics of observed lesions, genetic profile, and/or any other risk factor) to determine (e.g., include or exclude) whether a patient should be tested using an invasive technique. Accordingly, a clinical algorithm may have different cutoff or threshold levels for excluding patients with different risk profiles.

According to aspects of the invention, in order to detect indicia of disease in colonic effluent or stool detection methods may involve techniques that can detect nucleic acids that are present at 10%, 1%, 0.1%, 0.01%, 0.01%, 0.001% or lower frequencies amongst nucleic acid (for example human DNA, e.g. cell-free human DNA) in stool or colonic effluent. High sensitivity and/or high specificity assays may be particularly useful when screening a population of subjects and when clinical detection sensitivities of at least 60%, 65%, 70%, 75%, 80%, 85%, 90%, 91%, 92%, 93%, 94%, 95%, 96%, 97%, 98%, 99%, or higher are desired.

In one aspect, methods of the invention involve isolating and/or assaying a threshold number of genome equivalents (a genome equivalent of a particular genetic locus is a number of copies of that locus that are present in a single genome) of one or more predetermined genetic loci. Each genetic locus may be isolated as part of a target molecule as described herein. In certain embodiments, a target molecule may be isolated as a molecule of a predetermined size (or a predetermined minimal size) that contains at least the genetic locus of interest). For example, in some embodiments, a target nucleic acid molecule (either single-stranded or double-stranded) may be at least 200 bp, at least 300 bp, at least 400 bp, at least 500 bp, at least 600 bp, at least 700 bp, at least 800 bp, at least 900 bp, at least 1 kb, at least 1.1 kb, at least 1.2 kb, at least 1.3 kb, at least 1.4 kb, at least 1.5 kb, at least 1.6 kb, at least 1.7 kb, at least 1.8 kb, at least 1.9 kb, at least 2 kb, at least 2.1 kb, at least 2.2 kb, at least 2.3 kb, at least 2.4 kb, at least 2.5 kb long, or longer. As explained herein, when isolating a threshold number of genome equivalents of a particular locus, the predetermined threshold number may be determined by the desired sensitivity (either the assay sensitivity for detecting at least a predetermined low frequency abnormal nucleic acids and/or the clinical sensitivity for detecting at least a predetermined percentage of diseased individuals). In some embodiments, at least 100, at least 200, at least 300, at least 400, at least 500, at least 600, at least 700, at least 800, at least 900, at least 1000, at least 1100, at least 1200, at least 1300, at least 1400, at least 1500, at least 1500 to 2000, at least 2000 to 5000, at least 5000 to 10000, at least 10000 to 50000, at least 50000 to 100000, at least 100000 to 500000, or more genome equivalents of each genetic locus being analyzed may be isolated and/or introduced into a molecular assay. The number of genome equivalents may be determined using a known number of genome equivalents as a reference in an assay such as a real time PCR, a quantitative PCR, or other technique suitable for evaluating the number of copies of a particular genetic locus that may be present in a colonic sample.

In one embodiment, a high number of genome equivalents may be obtained by using an appropriate amount of starting material. The amount of starting material may be determined, at least in part, by the efficiency of the isolation procedure.

In one embodiment, a high number of genome equivalents may be isolated by using a high-efficiency purification method. For example, a hybrid capture method using a probe that binds to the target nucleic acid(s) may be used. In one embodiment, the hybrid capture method may involve attaching capture probe(s) to an electrophoretic medium and electrophoresing a biological sample over the immobilized capture probe. The electrophoretic medium may be agarose, polyacrylamide, beads, other configurations of suitable polymeric material, or combinations thereof. In some embodiments, a sample containing target nucleic acid may be exposed repeatedly to an immobilized capture probe (e.g., by electrophoresing the sample over the immobilized capture probe two or more times, for example using reversed phase electrophoresis).

In aspects of the invention, one or more different genetic abnormalities associated with a disease may be assayed for. Genetic abnormalities may be one or more specific mutations, or mutations in specific regions (mutations may be point mutations, insertions, deletions, duplications, inversions, translocations, etc.) known to be associated with the disease of interest. However, in some embodiments a genetic abnormality may be a nucleic acid characteristic of disease such as the presence of abnormally long nucleic acid or an abnormal amount of long (e.g., longer than 200 bp, longer than 500 bp, longer than 750 bp, longer than 1000 bp, longer than 1500 bp, longer than 2000 bp, or longer) nucleic acid in a stool sample or a colonic effluent sample. This assay, referred to as a DNA integrity assay, is described herein and in the art. In this assay, the presence of any long DNA above a threshold size or amount may be indicative of the presence of disease (e.g., adenoma, cancer, precancer, etc.). Any locus may be interrogated. However, in certain embodiments a locus that also may contain a particular mutation of interest is interrogated in a DNA integrity assay. In this embodiment, if the DNA integrity assay is positive, then the large nucleic acid may be interrogated for a mutation of interest.

In some embodiments, in order to increase the sensitivity of an assay for detecting one or more mutations, a detection analysis may be performed on target nucleic acid of a size above a threshold size that is positive for a DNA integrity assay (e.g., longer than 200 bp, longer than 500 bp, longer than 1000 bp, longer than 1500 bp, or longer). In some embodiments, this may be accomplished by first amplifying target nucleic acid using primers that bind to sequences on the target nucleic acid that are spaced apart by at least a predetermined threshold size. In certain embodiments, a threshold sized nucleic acid may be interrogated by using a capture probe that binds to a captured sequence on a target nucleic acid, wherein the captured sequence is at least a predetermined threshold distance from the target site for which a sequence determination or detection assay may be performed. In addition, or alternatively, aspects of the invention may involve one or more other methods described herein.

Markers indicative of the presence of a disease can be detected by using any method known in the art, including by reference to a nucleotide database, such as GenBank, EMBL, or any other appropriate database, by gel electrophoresis, or by other standard methods. In some embodiments, the regions considered are regions in which loss of heterozygosity is prevalent, such as regions containing tumor suppressor genes.

In general, any set of markers that can identify the presence of a disease can be used. In some embodiments, the marker set has a sensitivity of at least 50%, 75%, at least 80%, 85%, 90%, 95%, 98%, or 99%. The term “sensitivity” relates to the incidence of false negative results, i.e., to the probability that an individual in which a given mutation is present will be correctly identified. A test which has “high sensitivity” has few, e.g., fewer than 1% false negative results, and thus will rarely if ever miss the presence of a mutation, although it may provide an incorrect diagnosis for the presence of the mutation. For comparison, the term “specificity” relates to the incidence of false positive results in a particular tests, or stated differently to the probability that an individual in which a given marker is absent will be correctly identified. A test which has “high specificity” has few, e.g., fewer than 1% false positive results, and thus rarely, if ever, gives an erroneous indication that a mutation is present, but may fail to detect the mutation in some or even many instances. Any marker that will reveal the presence of a disease can be used. Markers are typically associated with alterations or mutations associated with the occurrence of a given disease (e.g. cancer, precancer, adenoma). Such mutations include, e.g., single or multiple basepair substitutions, single or multiple base pair insertions, and single or multiple basepair deletions. The marker set is selected so that it will be informative for a disease of interest. When the disease is colorectal cancer, a suitable marker set is one or more of the multi-target assay panel (MAP) described in Tagore et al., Clin. Colorectal Cancer 3:47-53, 2003, the contents of which are incorporated herein by reference in their entirety. The MAP includes specific mutations in the adenomatous polyposis coli (AFC), p53, and K-ras genes, a microsatellite instability marker 15 (BAT-26), and a marker of abnormal apoptosis (DNA Integrity Assay).

In general, any molecular assay can be used, provided that it is capable of detecting a marker associated with the disease in the sample tested. Preferred assays are those that are sensitive enough to detect rare nucleic acids in a population of nucleic acid molecules. A suitable assay can be, e.g., allele-specific PCR (Rano et al., Nucl. Acids Res. 17:8392, 1989) or mutation amplification mismatch assay (MAMA). In the MAMA-PCR method, one of the two PCR primers, the ‘mismatch detection’ primer, has two mismatched bases at the Y end with respect to the wild-type sequence (ultimate and penultimate T base); but a single mismatch with the mutated allele (the penultimate 3′ base). The two mismatched bases at-the 3′ end of the primer, when annealed to the wild-type template, fail to amplify a PCR product. However, in the case of the mutant DNA, the primer anneals to the template and allows selective amplification and detection of the targeted clone. Cha et al., PCR Methods Appl. 2: 14-20, 1993; Glaab et al., Mutat. Res. 430: 1-12, 1993. Other assays may be methylation detection assays (e.g. methylation specific PCR) capable of detecting hypermethylated genomic regions associated with disease.

Accordingly, aspects of the invention may be used to increase the sensitivity of a virtual colonoscopy. In some embodiments, a patient who is identified as negative in a virtual colonoscopy may be identified as positive in an adjunct molecular assay (e.g., on a sample of colonic effluent, stool, or both). This patient can be re-evaluated by virtual colonoscopy, additional molecular testing, invasive testing, or any combination of two or more thereof. It should be appreciated that molecular analytical techniques and compositions described herein also may be used as an adjunct to an invasive assay such as a colonoscopy or a sigmoidoscopy to provide further specificity and/or sensitivity.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. Other features and advantages of the invention will be apparent from the following detailed description and claims.

### DETAILED DESCRIPTION OF THE INVENTION

Aspects of the invention relate generally to the use of clinical algorithms for excluding patients identified in virtual imaging. Virtual imaging may be used to identify a patient as one requiring invasive testing. In aspects of the invention, methods of obtaining and analyzing a colonic effluent to determine if a patient requires invasive testing are provided.

Aspects of the invention relate to a clinical algorithm using a molecular detection assay as an adjunct to virtual imaging to exclude a fraction of image-positive subjects from subsequent invasive testing.

In particular, aspects of the invention relate to performing a molecular assay on colonic effluent collected during a virtual colonoscopy (e.g., a CT colonoscopy) in order to exclude a subset of patients from subsequent invasive testing (e.g. colonoscopy). Aspects of the application relate to screening patients that were identified as positives during the virtual colonoscopy due to a visual observation of abnormalities in their colon. In a subset of these patients, the positive visual detection is a non-cancerous lesion at one or more positions along the colon. In order to identify this subset of patients and exclude them from subsequent invasive testing (e.g. invasive colonoscopy and/or biopsy), one or more molecular assays may be performed on the colonic effluent.

Examples of non-cancerous lesions that may be identified in a virtual colonoscopy as candidates for subsequent invasive colonoscopy and biopsy include benign polyps, ulceration, scarring, certain inflammatory conditions, other epithelial disruptions or disruptions of the architecture of the colonic lining. An assay that excludes these patients from the invasive testing reduces clinical cost, patient discomfort, and patient risk.

However, it should be appreciated that in some embodiments it may be preferable to include some healthy patients rather than exclude diseased patients.

Threshold signal levels for the molecular assays may be determined to prevent or minimize the exclusion of diseased conditions (e.g., cancer, pre-cancer, adenoma, or any malignant growth or malignant tumor). For example, the assays may have a sensitivity of over 50%, e.g. over 60%, over 70%, or about 80%, about 85%, about 90%, about 95%, about 96%, about 97%, about 98%, about 99%, or higher, e.g., up to 100%. In preferred embodiments, a molecular assay has a tissue sensitivity of greater than 85%, greater than 90%, e.g. about 91%, about 92%, about 93%, about 94%, about 95%, about 96%, about 97%, about 98%, about 99%, about 100%.

Aspects of the invention relate to preserving a colonic effluent sample. Accordingly, the colonic effluent may be mixed with a stabilization buffer. In one embodiment, it may be directly aspirated into a container with stabilization buffer.

In some aspects, nucleic acid may be captured from the colonic effluent (using, for example, hybrid capture). In some embodiments, cells in a colonic effluent may be lysed prior to nucleic acid isolation (e.g., by hybrid capture).

In some aspects, an assay may include interrogating a minimum number of genome equivalents (for example, 100, 200, 300, 400, and preferably 500 or more, e.g., 1000, 1500, 2000, 2500, or more) to achieve a high level of informativeness. In some aspects, a patient for which a low number of genome equivalents are recovered may be designated for a subsequent invasive analysis even if the molecular analysis is negative.

According to aspects of the invention, colonic effluent may be the material obtained from the colon, before, during or after a virtual colonoscopy. Colonic effluent material may obtained as a solution resulting from a colonic lavage or an enema. The material may be obtained prior to, during, or after a virtual colonoscopy, or any combination thereof. The colonic effluent may contain particulates from the colon dispersed in any liquid or solution used in association with the virtual colonoscopy. In some aspects, particulate matter may be removed from a colonic effluent, for example using centrifugation. In some embodiments, water may be used to generate a colonic effluent. However, in certain embodiments, a liquid or solution used to generate a colonic effluent may be an aqueous solution that contains one or more of a buffer, a salt, an alcohol, a solvent, a viscous material, an agent that stabilizes nucleic acids, etc., or any combination thereof.

Virtual Imaging

Aspects of the invention relate to virtual imaging procedures. In some embodiments, virtual imaging may be used to identify a patient suspected of having colon cancer or other colon abnormality. In certain embodiments, virtual imaging includes, but is not limited to, gastrointestinal imaging. In some embodiments, gastrointestinal imaging includes, but is not limited to, X-ray imaging or other virtual gastrointestinal imaging. As an example, virtual gastrointestinal imaging includes any technique involving the use of computer software to view any internal section of a gastrointestinal tract. Virtual gastrointestinal imaging can be, but is not limited to, CT imaging, MR imaging, VCT (volumetric computer-assisted tomography) or PET imaging (positron emission tomography).

Aspects of the invention relate to a clinical algorithm using a molecular detection assay as an adjunct to virtual imaging to exclude a patient from requiring invasive testing. In some embodiments, a fraction of image-positive subjects are excluded from subsequent invasive testing. In certain embodiments, a fraction includes, but is not limited to, patients identified as positive having borderline positive virtual images. Such a patient may be determined by those of ordinary skill in the art.

Aspects of the invention relate to performing a molecular assay on colonic effluent collected during a virtual colonoscopy (e.g., a CT colonoscopy) in order to exclude a subset of patients from subsequent invasive colonoscopy. In some embodiments, the invention relates to screening patients that were identified as positives during the virtual colonoscopy due to the visual observation of abnormalities in their colon. In a subset of these patients, the positive visual detection is a non-cancerous lesion at one or more positions along the colon. In order to identify this subset of patients and exclude them from subsequent invasive biopsy testing, one or more molecular assays may be performed on the colonic effluent.

A colonic effluent may be collected prior to, during, or after a virtual imaging procedure. Colonic effluent collected at any stage may be analyzed to exclude false positive patients and to further identify positive patients. The analysis of the colonic effluent allows the exclusion of false positive patients and prevents them having to undergo invasive procedures such as a colonoscopy.

In aspects of the invention, the colonic effluent of a patient identified as positive by virtual imaging may be analyzed using any method as described herein. The colonic effluent may be analyzed using for example, single molecule sequence analysis technology (e.g., sequencing technology that was developed for whole genome sequence analysis).

In certain embodiments, indicia of disease (e.g., one or more genetic abnormalities including one or more point mutations, insertions, deletions, duplications, inversions, translocations, and/or other genetic abnormalities associated with the presence of disease) may be detected in a colonic sample suspected of containing a high amount of abnormal cells or nucleic acid relative to normal cells and nucleic acid and/or a heterogeneous colonic sample suspected of containing relatively few abnormal cells and/or nucleic acid (e.g., cell free DNA) amongst an abundance of normal cells and/or nucleic acid. It should be appreciated that relatively more abnormal nucleic acid (nucleic acid with one or more genetic abnormalities) will be present in a biopsy of tissue that is diseased than in a heterogeneous biological sample that does contain small amounts of disease-associated abnormal nucleic acid or cells. Therefore, detection assays with high sensitivity and specificity may be more important for the analysis of heterogeneous samples than for biopsy samples of diseased tissues. However, high sensitivity and high specificity assays also may be used for analyzing biopsy samples as the invention is not limited in this respect. High sensitivity detection assays, high specificity detection assays, high efficiency nucleic acid capture methods, nucleic acid stabilization methods, and other methods useful for isolating and/or detecting rare nucleic acids are described in more detail herein.

In some embodiments, a molecular assay involves interrogating a plurality of genetic loci (e.g., disease associated markers) that may be present on a plurality of different target nucleic acids. For example, 5-10, 10-15, 15-20, 20-25, 25-30, or more different loci may be interrogated for the presence of any combination of mutations (e.g., point mutations, insertions, deletions, duplications, translocations, etc.) In certain embodiments, the assay interrogates genetic loci that have a sensitivity for detecting the disease (e.g., adenoma, cancer, precancer, etc.) in a subject of at least 50%, 75%, 80%, 85%, 90%, 95%, 98%, or 99%.

As discussed herein, due to the stochastic nature of certain isolation and analytical techniques (particularly amplification techniques, such as PCR) it may be necessary to use isolation, stabilization and/or capture methods that provide at least a threshold amount of genome equivalents of a target nucleic acid so that any measurement of the amount of abnormal nucleic acids is statistically significant. It should be appreciated that higher amounts of genome equivalents (a genome equivalent of a genetic locus is an amount of that locus that is present in a genome of the subject) of one or more predetermined genetic loci will provide more statistically significant and informative results.

It also should be appreciated that the ability to detect a molecular indicia early stage diseases (e.g., adenomas, precancerous growths, etc.) may be improved with the use of high specificity and sensitivity assays, high efficiency nucleic acid capture methods and devices, nucleic acid preservation methods and compositions, and/or other methods and compositions useful for the detection of rare abnormal nucleic molecules against a background of abundant normal nucleic acid molecules. In one embodiment, a high complexity and high sensitivity assay may be used for detection. However, in other embodiments, a low complexity and high sensitivity or a low complexity and low sensitivity assay may be used depending on the scope of genetic abnormalities and are being assayed for and the type of tissue that is being analyzed (e.g., if the colonic effluent is enriched for abnormal nucleic acid relative to a stool sample). These and other methods are described in more detail herein.

An assay may include interrogating a minimum number of genome equivalents (for example, 100, 200, 300, 400, and preferably 500 or more, e.g., 1000, 1500, 2000, 2500, or more) to achieve a high level of informativeness. In some aspects, a patient for which a low number of genome equivalents are recovered may be designated for a subsequent invasive analysis even if the molecular analysis is negative.

In one aspect, methods are provided for detecting one or more particular mutations that are known to be associated with certain diseases or for detecting mutations at one or more genetic loci known to be associated with certain diseases. In certain embodiments, methods that can detect the presence of one or more mutations in particular target regions may be used. Useful methods include assays that can detect small amounts of mutant nucleic acid in a background of normal nucleic acid.

In certain aspects, DNA may be isolated from a colonic effluent. Isolation of DNA may be performed using any methods known to those of skill in the art, for example using a commercial kit that provides the required buffers and instructions to isolate DNA from any sample, including a colonic effluent. Those of skill in the art are familiar with such kits.

In aspects of the invention, molecular assays may be used in the analysis of colonic effluent. Examples of molecular assays include: DIA, multiple mutation analysis, LOH, methylation analysis, or a combination thereof, including, for example, analyzing one or more predetermined marker panels.

Certain aspects of the invention relate to combining single molecule sequence analysis technology (e.g., sequencing technology that was developed for whole genome sequence analysis) with specific sequence capture technology in order to detect rare genetic abnormalities at one or more genetic loci. Accordingly, aspects of the invention allow isolation and detection of very low frequency nucleic acid molecules having rare genetic abnormalities by combining i) a high efficiency specific sequence capture step that yields a nucleic acid preparation of relatively low genomic complexity containing several genome equivalents of a target nucleic acid of interest with ii) a high complexity analytical step, such as single molecule sequence analysis that can be used to characterize (e.g., sequence) each of a plurality of genome equivalents of the target nucleic acid. According to the invention, a high complexity analytical step may be used to detect (for example, with statistically significant confidence, e.g., greater than 90%, greater than 95%, or greater than 99% confidence) the presence or absence of a rare nucleic acid in a preparation of captured nucleic acid molecules having identical or substantially identical sequences.

Aspects of the invention relate to methods for detecting indicia of diseases (e.g., adenomas and/or early stage cancers) in biological samples. In particular, aspects of the invention relate to methods for detecting the presence of rare altered/mutant nucleic acid molecules that are present at a very low frequency in a biological sample containing a majority of normal nucleic acid molecules. According to the invention, altered/mutant nucleic acid indicative of adenoma and/or early stage cancer and/or other diseases may be present only at a frequency of less than 1% (e.g., less that 0.1%) of the total genome equivalents in a biological sample. Aspects of the invention are useful for both isolating and detecting such rare nucleic acid molecules. According to the invention, a detection assay may fail to detect nucleic acid molecules that are present at a very low frequency in a biological sample if either i) a capture step fails to capture a rare nucleic acid molecule that is present in a biological sample and/or ii) a detection reaction fails to detect a rare nucleic acid that is present in a preparation of captured nucleic acid.

According to aspects of the invention, the captured nucleic acid molecules may be relatively small, for example, from about 50 bases long to about several kilo-bases long (e.g., between about 100 bases and 10,000 bases, or about 150 bases, about 200 bases, about 250 bases, about 300 bases, about 350 bases, about 400 bases, about 450 bases, about 500 bases, about 1,000 bases, about 1,500 bases, about 2,000 bases, about 2,500 bases, about 3,000 bases, about 5,000 bases long, etc.). However, longer or shorter nucleic acid molecules may be captured. A typical biological sample may contain (or be processed to contain) nucleic acid fragments distributed across a range of sizes such as those described above. It should be noted that genomic nucleic acid in certain biological samples (e.g., stool samples) is already fragmented with typical fragment sizes ranging from 50 bases to several hundred bases long. A captured nucleic acid may be single stranded, double stranded, or contain both single and double-stranded regions. A captured nucleic acid may be DNA, RNA, or a modified form thereof. Nucleic acid may be captured from the colonic effluent (using for example hybrid capture, e.g. repetitive reversed-field hybrid capture electrophoresis).

It should be appreciated that aspects of the invention described herein, although particularly useful for screening for adenomas or early stage cancer, also may detect later stage cancers. An assay with sufficient sensitivity to detect adenomas or early stage cancer will be sufficiently sensitive to detect altered/mutant nucleic acid from a later stage cancer that is present at a higher frequency in a colonic sample. Similarly, aspects of the invention may be used to screen for molecular markers of other diseases that are associated with the presence of abnormal nucleic acid in a colonic sample. Aspects of the invention may be used to detect the presence, in a colonic sample, of nucleic acid abnormalities associated with other diseases. Other diseases may include one or more inflammatory conditions, infections (including, for example, intracellular viral modifications), etc.

Detecting Rare Abnormal Nucleic Acid Molecules in Biological Samples:

Digital and/or High Complexity Sequence Analysis

Any of the assays described herein may be performed in a digital format wherein a sample is diluted into aliquots wherein each aliquot contains on average between 1 and 20 target nucleic acid molecules (e.g., DNA) for analysis (e.g., between 1 and 10 molecules, between 1 and 5 molecules, on average 1, etc.).

Aspects of the invention may include analyzing a predetermined number of genome equivalents of one or more target nucleic acids in order to determine whether one or more of the individual target nucleic acid molecules contains an abnormal sequence.

In aspects of the invention, the presence of a low frequency altered/mutant target nucleic acid molecule in a captured preparation of target nucleic acid molecules of low genomic complexity may be detected using a technique that was designed for analyzing nucleic acid samples of high genomic complexity. In some aspects of the invention, methods for sequencing whole genomes or substantial portions thereof (e.g., chromosomes or significant portions thereof) may be used to detect low frequency events in a nucleic acid sample of low genomic complexity.

High complexity analytical techniques may involve primer extension (e.g., single base extension or multiple base extension) or nucleic acid detection techniques that can analyze large numbers of different template nucleic acid molecules (e.g., sequence or provide the identity of at least one nucleotide position in a template molecule). High complexity analytical techniques may involve the parallel and/or serial processing of a large number of different template nucleic acid molecules. High complexity analytical techniques may involve a parallel and/or serial analysis of single molecules (e.g., single nucleic acid molecule sequencing). In one aspect, a preparation of template molecules may be dispersed across a solid surface and individual molecules may be immobilized on the surface (e.g., a microscope slide or similar substrate). A high sensitivity analytical technique may be used to characterize each immobilized molecule individually. For example, primer extension reactions may be used to incorporate labeled nucleotide(s) that can be individually detected in order to sequence individual molecules and/or determine the identity of at least one nucleotide position on individual template nucleic acid molecules. Detection may involve labeling one or more of the primers and or extension nucleotides with a detectable label (e.g., using fluorescent label(s), FRET label(s), enzymatic label(s), radio-label(s), etc.). Detection may involve imaging, for example using a high sensitivity camera and/or microscope (e.g., a super-cooled camera and/or microscope).

Accordingly, a “high complexity analytical step” may be a process that can analyze nucleic acid preparations of high genomic complexity. According to the invention, a preparation of target nucleic acid molecules of low genomic complexity such as those described herein may be used as template molecules and processed using a high complexity analytical technique. According to the invention, a high complexity analytical technique may be used to detect rare mutant/altered nucleic acid molecules in a preparation of many similar (or identical) nucleic acid templates of low genomic complexity.

Examples of high complexity nucleic acid analytical techniques are described herein. Additional analytical techniques are known in the art. Suitable techniques may be selected by one of ordinary skill in the art using the teachings of the invention. According to the invention, a sufficient number of target molecules should be captured and analyzed. A sufficient number is a number that provides a statistically significant result (e.g., a confidence level of at least 80%, at least 90%, at least 95%, or at least 99% that a particular alteration or mutation is either present or absent from a biological sample being analyzed).

In one embodiment, a digital analysis (e.g., a digital amplification and subsequent analysis) may be performed on at least a sufficient number of molecules to obtain a statistically significant result. Certain digital techniques are known in the art, see for example, U.S. Pat. No. 6,440,706 and U.S. Pat. No. 6,753,147, the entire contents of which are incorporated herein by reference. Similarly, an emulsion-based method for amplifying and/or sequencing individual nucleic acid molecules may be used (e.g., BEAMing technology).

In one embodiment, a sequencing method that can sequence single molecules in a biological sample may be used. Sequencing methods are known and being developed for high throughput (e.g., parallel) sequencing of complex genomes by sequencing a large number of single molecules (often having overlapping sequences) and compiling the information to obtain the sequence of an entire genome or a significant portion thereof. According to the invention, such methods, although designed for complex sequence analysis, may be particularly suited to sequence a large number of substantially identical molecules in order to identify the rare one(s) that contain a mutation or alteration.

High complexity analytical or sequencing techniques may involve high speed parallel molecular nucleic acid sequencing as described in PCT Application No. WO 01/16375, U.S. Application No. 60/151,580 and U.S. Published Application No. 20050014175, the entire contents of which are incorporated herein by reference. Other sequencing techniques are described in PCT Application No. WO 05/73410, PCT Application No. WO 05/54431, PCT Application No. WO 05/39389, PCT Application No. WO 05/03375, PCT Application No. WO 05/010145, PCT Application No. WO 04/069849, PCT Application No. WO 04/70005, PCT Application No. WO 04/69849, PCT Application No. WO 04/70007, and US Published Application No. 20050100932, the entire contents of which are incorporated herein by reference.

High complexity analytical or sequencing techniques may involve exposing a nucleic acid molecule to an oligonucleotide primer and a polymerase in the presence of a mixture of nucleotides. Changes in the fluorescence of individual nucleic acid molecules in response to polymerase activity may be detected and recorded. The specific labels attached to each nucleic acid and/or nucleotide may provide an emission spectrum allowing for the detection of sequence information for individual template nucleic acid molecules. In certain embodiments, a label is attached to the primer/template and a different label is attached to each type of nucleotide (e.g., A, T/U, C, or G). Each label emits a distinct signal which is distinguished from the other labels.

High complexity analytical or sequencing techniques may involve or be based on methods or technology described in Shendure et al., Nature Reviews/Genetics, Volume 5, May 2004, pages 335-344; Braslavsky et al., PNAS, Apr. 1, 2003, Volume 100, No. 7, pages 3960-3964; the entire disclosures of which are incorporated herein by reference.

In other embodiments, high complexity analytical or sequencing techniques may involve providing a primed target polynucleotide linked to a microfabricated synthesis channel, and flowing a first nucleotide through the synthesis channel under conditions such as to allow the first nucleotide to attach to the primer. The presence or absence of a signal is determined, the presence indicating that the first nucleotide was incorporated into the primer and the identity of the complementary base that served as a template in the target polynucleotide is determined. The signal is then removed or reduced and the process repeated with a second nucleotide. The second nucleotide can be either the same as the first nucleotide or a different nucleotide. The specific labels attached to each nucleic acid provide an emission spectra allowing for detection of sequence information of the nucleic acid molecule. In other embodiments, a plurality of different primed target polynucleotides linked to different synthesis channels is used. In further embodiments, the polynucleotide is attached to a surface. In some embodiments, a label is attached to the nucleotide.

In certain embodiments, a high complexity analytical or sequencing technique may be provided by Helicos BioSciences Corporation (Cambridge, Mass.). Briefly, in some embodiments, single strands of purified DNA with a universal priming sequence at each end of the strand may be generated. The strands are labeled with a fluorescent nucleotide and hybridized to primers immobilized on a surface. The primer duplexes are analyzed and the positions of each duplex recorded. DNA polymerase and a fluorescently labeled nucleotide are added and bind the appropriate primers. The sample is washed to remove unbound nucleotides and excess polymerase. The samples are analyzed and the positions of the incorporated nucleotides recorded. The fluorescent label is removed and a second labeled nucleotide is added and the process is repeated. The process may be repeated several times until a desired length is reached.

Other useful genome/complex sequencing methods include high throughput sequencing using the 454 Life Sciences Instrument System. Briefly, a sample of single stranded DNA is prepared and added to an excess of DNA capture beads which are then emulsified. Clonal amplification is performed to produce a sample of enriched DNA on the capture beads (the beads are enriched with millions of copies of a single clonal fragment). The DNA enriched beads are then transferred into PicoTiterPlate™ and enzyme beads and sequencing reagents are added. The samples are then analyzed and the sequence data recorded. Pyrophosphate and luciferin are examples of the labels that can be used to generate the signal.

A label may be, but is not limited to, a fluorophore, for example green fluorescent protein (GFP), a luminescent molecule, for example aequorin or europium chelates, fluorescein, rhodamine green, Oregon green, Texas red, naphthofluorescein, or derivatives thereof. In some embodiments, the polynucleotide is linked to a substrate. A substrate may be, but is not limited to, streptavidin-biotin, histidine-Ni, S-tag-S-protein, or glutathione-S-transferase (GST). In some embodiments, a substrate is pretreated to facilitate attachment of a polynucleotide to a surface, for example the substrate can be glass which is coated with a polyelectrolyte multilayer (PEM), or the polynucleotide is biotinylated and the PEM-coated surface is further coated with biotin and streptavidin.

In other embodiments, single molecule sequencing technology available from US Genomics, Woburn, Mass., may be used. For example, technology described, at least in part, in one or more of U.S. Pat. Nos. 6,790,671; 6,772,070; 6,762,059; 6,696,022; 6,403,311; 6,355,420; 6,263,286; and 6,210,896 may be used.

Other sequencing methods, including other high complexity analytical techniques also may be used to analyze DNA and/or RNA according to methods of the invention. It should be appreciated that a sequencing method does not have to be a single molecule sequencing method. In one embodiment, a method that sequences small numbers of molecules (e.g., 2, 3, 4, 5, 6, 7, 8, 9, 10, to about 15 or about 20 molecules) in a single reaction may be useful if the results can reliably detect the presence of a single (or a small number) of abnormal nucleic acids amongst the number of molecules that are being sequenced. It should be appreciated that the entire sequence of a captured target molecule does not need to be determined. It is sufficient to determine the sequence at a position, on the target molecule, suspected of containing an abnormality. It also should be appreciated that the analytical method does not need to identify the actual sequence of each molecule. In some embodiments, it may be sufficient to detect the presence of a small number (e.g., one or two) or a small percentage (e.g., 10%, 5%, 1%, 0.1%, 0.01% or lower) of abnormal molecules in a sample. For example, certain physical characterizations (e.g., mass detection such as mass spectrometry) may be used to distinguish normal from abnormal molecules and detect the presence of a small amount of abnormal nucleic acids associated with a disease.

Using Two or More Polymerases in Primer Extension Reactions

In certain aspects of invention, rare abnormal nucleic acids may be detected and/or characterized using primer extension reactions performed using two or more different polymerases for detecting and/or identifying mutant nucleic acids within a heterogeneous population of nucleic acids. In one embodiment, methods of the invention reduce misincorporation of terminator nucleotides (e.g., in single base-scanning reactions) by providing a first polymerase that preferentially incorporates extending nucleotides and a second polymerase that preferentially incorporates terminating nucleotides. In one embodiment, one or both of the polymerases misincorporate(s) an incorrect nucleotide (e.g., a terminating nucleotide) with a frequency of less than about 10%, for example less than about 5%, less than about 1%, or less than about 0.1%. It should be appreciated that misincorporation of a terminating nucleotide at a non-complementary position in an extension reaction can generate a false positive indication of the presence of a mutation at the site of misincorporation. According to the invention, a significant source of misincorporation may result from a correctly hybridized primer being extended with an incorrect nucleotide (i.e., a nucleotide that is not complementary to the template at the position where it is incorporated into the primer extension product).

In one aspect, misincorporation may be reduced by performing a primer extension reaction (e.g., a single base-scanning reaction) in the presence of at least two polymerases, each of which preferentially incorporates one of two nucleotides: an extending nucleotide or a terminating nucleotide. By including different polymerases for the extending and terminating nucleotides, polymerases that have low frequencies of incorrect base incorporation may be used. Preferably, one of the nucleotides is labeled and the other one is not labeled. The labeled nucleotide is preferably the one that is incorporated in the primer extension product when the template contains a sequence to be detected. In alternative embodiments, both first and second nucleotide may be labeled if they are differentially labeled such that the label on one of the nucleotides is detectably different from the label on the other nucleotide.

According to the invention, reducing the rate of misincorporation increases the sensitivity of primer extension assays (e.g., single base scanning assays) and allows for the detection of rare nucleic acid variations in heterogeneous biological samples. Misincorporation of a nucleotide corresponding to a mutation in a primer extension reaction can lead to a false positive detection of the presence of the mutation in a nucleic acid sample. In a typical primer extension reaction on a biological sample that contains mostly wild-type nucleic acids, misincorporation results in a background level of false positive signal that is high enough to obscure a true positive signal generated from a relatively small amount of mutant nucleic acids. Therefore, by reducing the amount of misincorporation, aspects of the invention provide highly sensitive and highly specific assays for detecting rare variant and/or mutant nucleic acid in heterogeneous biological samples.

In one aspect of the invention, single base scanning methods include identifying a target nucleic acid region suspected of containing a variation, and interrogating the target region using a single base scanning reaction. A primer is hybridized to a single stranded nucleic acid in the presence of extension nucleotides and polymerases, and the primer is extended through the target region creating primer extension products that are subsequently detected and/or quantified.

As discussed herein, different polymerases characterized by preferential nucleotide incorporation may be used to increase the signal to noise ratio for detecting low frequency events (e.g., mutant nucleic acids that are present at a low frequency in a biological sample containing an excess of non-mutant nucleic acids). In one embodiment, a first polymerase preferentially incorporates the extending nucleotide that is used, and a second polymerase preferentially incorporates the terminating nucleotide that is used. Preferential incorporation of one nucleotide over another can be measured using any suitable technique, for example by running parallel reactions which differ only in the type and concentration of each nucleotide. The relative incorporation efficiency of two different nucleotides can be reflected in the concentrations of nucleotides in the two reactions. For example, a first reaction that contains 10-fold more of a first nucleotide than a second reaction containing a second nucleotide demonstrates that insertion of the first nucleotide is 10-fold less efficient than that of the second nucleotide. These concentration levels can be measured by various methods, including, for example, performing titration assays, running the samples on DNA sequencing gels and visualizing the extent of nucleotide incorporation by autoradiography, running capillary electrophoresis assays and determining the levels of nucleotide incorporation. In one embodiment, if the incorporation of a first nucleotide over a second nucleotide is a 10-fold or greater difference, a determination can be made that the first terminator was preferentially incorporated. Preferably, a first polymerase incorporates a first nucleotide with between a 2 fold and a 100 fold preference. In certain embodiments, the ratio of incorporation may be between 5 fold and 50 fold, for example the ratio may be about 10 fold. However, ratios of less than 2 fold and greater than 100 fold can also be useful. The preference of the first polymerase for the first nucleotide relative to the second nucleotide can also be measured as a percentage increase in incorporation in an assay. Preferably, a first polymerase has between about a 5% and a 100% preference for a first type of nucleotide relative to a second type of nucleotide, for example between about 25% and about 75%. Examples of DNA polymerases with preferential nucleotide incorporation properties include those that preferentially incorporate dideoxy terminators over acyclic-terminators include Taq polymerase and Thermo Sequenase. (Gardner, A., (2002) “Acyclic and dideoxy terminator preferences denote divergent sugar recognition by archaeon and Taq DNA polymerases”, Nucleic Acids Research, Vol. 30, No. 2 pp. 605-613.) Examples of DNA polymerases that preferentially incorporate acyclic terminators include Vent, Vent A488L, Deep Vent, 9°Nₘ, Pfu, and AcycloPol. In one embodiment of the invention, preferential incorporation can also be achieved by using dye-labeled terminators. Examples of DNA polymerases that preferentially incorporate dye-labeled terminators include Vent DNA polymerase, which preferentially incorporates dye-labeled dCTP analogs over unmodified dCTPs, and dye-acyCTPs over dye-ddCTPs, and Vent, Deep Vent, Pfu and 9°Nₘ polymerases, which preferentially incorporate dye-acyNTPs over dye-ddNTPs. Similarly, preferential incorporation of terminator nucleotides relative to extending nucleotides or extending nucleotides relative to terminator nucleotides may be assessed and polymerases with preferential incorporation properties may be used as described herein.

Preferential Analysis and/or Characterization of Abnormal Nucleic Acids

Aspects of the invention may include a step that preferentially isolates abnormal nucleic acid molecules from a sample that includes both normal and abnormal nucleic acids. In one embodiment, PCR amplification can be performed in the presence of blocking oligonucleotides that suppress amplification of predetermined sequences in a population of nucleic acid sequences. The sequences whose amplification is blocked are typically those that are present in excess in a starting population of mixed nucleic acids. For example, if a sequence containing a mutation is present in a small amount in a population of nucleic acid sequences that do not contain the mutation, amplification of the latter sequences can be suppressed by adding blocking oligonucleotide or nucleotides prior to, or concurrently with, performing the PCR reaction. The blocking oligonucleotides preferably bind specifically (and in some embodiments, exclusively) to sequences not containing the mutation. The result is to increase the relative representation of the mutant sequence in a population of amplified sequences. A blocking oligonucleotide can be, e.g., a peptide nucleic acid (PNA), a locked nucleic acid (LNA), or a oligonucleotide including one or more phosphine analogues, PEGA modified antisense constructs 2′-O-methyl nucleic acids, 2′-fluoro nucleic acids, phosphorothioates, and metal phosphonates.

In one embodiment a DNA integrity assay (DIA) may be performed to detect the presence of long DNA fragments derived from abnormal cells (e.g., adenomas, cancerous or precancerous cells) that were shed into a lumen via a process that does not involve apoptosis. The DIA assay has been previously described in detail. The DIA assay involves detecting (e.g., in an amplification reaction such as a PCR reaction) the presence, in a biological sample from a lumen (e.g., a stool sample or a colonic effluent sample) of large DNA fragments (e.g., longer than about 200 bp, longer than about 500 bp, longer than about 1 kb, longer than about 1.5 kb, etc.) in an amount higher than expected (e.g., observed) for a healthy patient. In one embodiment, the amount of long DNA may be compared to a reference amount characteristic of a patient known to have a disease. More recently this assay has been converted to a real-time PCR methodology. In one embodiment, three unique PCR reactions (in duplicate) per loci may be run on I-Cycler instruments (BioRad; Hercules, Calif.). In one embodiment, a DIA method may involve capturing locus specific segments and performing small (e.g., ˜100 bp) PCR amplifications remote from the capture site as an indicators of DNA length. DNA fragments for integrity analysis may be amplified at any locus, for example from four different loci: 17p13; 5q21; HRMT1L1; LOC91199 (named DIA-D, DIA-E, DIA-X, and DIA-Y, respectively). PCR primer sets and associated TaqMan probe for each locus of interest may be “walked” down the chromosome thereby interrogating for the presence and quantitation of increasing length of DNA of approximately, 100 bp, 1300 bp, 1800 bp and 2400 bp fragments of captured DNA. In one embodiment, purified DNA template (5 μl) was mixed with, 5 μl 10×PCR buffer (Takara), 10 μl dNTP's (2 mM) (Promega), 0.25 μl LATaq (5 U/μl; Takara), 24.75 μl molecular biology grade water (Sigma), 5 μl of a mix of PCR primers (5 μM; Midland) and TaqMan dual-labeled probes (2 μM; Biosearch Technologies). The I-Cycler was programmed as follows: 94° C. for 5 minutes, then 40 cycles of 94° C. for 1 minute, 55° C. for 1 minute, and 72° C. for 1 minute. Genomic standards, prepared as 20, 100, 500, 2500, and 12,500 GE/5 μl were prepared and used to generate a standard curve. In one embodiment, threshold Genome Equivalents (GE) values were determined for each of 12 PCR reactions (corresponding to the 1.3 kb, 1.8 kb, and 2.4 kb fragments across the 4 genomic loci) using a previously determined set of cancers and normals. In one embodiment, a positive result may require at least 4 of the 12 PCR reactions to be above individual PCR thresholds in order to prospectively determine cancers. However, in other embodiments, different thresholds may be used.

It should be appreciated that long DNA is thought to originate from diseased cells. However, each long fragment of long DNA does not contain a genetic abnormality. Nonetheless, by interrogating one or more long DNA fragments (e.g., obtained using PCR primers separated by at least 200 bp, 500 bp, 1 kb, 1.5, kb or more) a sample may be enriched for nucleic acid sequences derived from diseased cells relative to normal cells. Accordingly, by interrogating a target nucleic acid (e.g., containing a region suspected of being abnormal in a diseased patient) of a length associated with abnormal cells, an increased signal to noise ratio of abnormal to normal may be obtained in an assay of the invention. Accordingly, interrogating longer DNA fragments may provide increased sensitivity for detecting rare abnormal (e.g., mutant) nucleic acids in a biological sample.

Sequence Scanning

In certain embodiments, the signal of mutant nucleic acid relative to normal nucleic acid may be enhance in a scanning reaction that interrogates a region for the presence of one or more mutations that may be associated with a diseased. In one aspect, current sequencing reactions may be modified such that only one terminator nucleotide (also referred to as a terminating nucleotide, i.e., a nucleotide that terminates an enzymatic primer extension reaction because it cannot be extended by a polymerase enzyme when it is incorporated into a primer extension product), and not all four terminator nucleotides, is provided in a primer extension reaction to allow for single base scanning, which is also referred to herein as single base tracking. The modified reaction is herein referred to as a single base tracking reaction. A single base tracking reaction of the invention may be used to detect the presence, in the nucleic acid region being scanned, of at least one aberrant nucleotide (e.g., a mutation, polymorphism, etc.) corresponding to the terminating nucleotide base being used in the scanning reaction. Aspects of the invention may be used to detect the presence of at least one variant nucleotide in at least one nucleic acid molecule being scanned even in the presence of an excess of nucleic acids that do not contain the variant nucleotide. Such an increased sensitivity has at least several uses. For example, methods according to the invention can be used to interrogate one or more nucleic acid regions for the presence of at least one genetic variation (e.g., mutation) in at least one nucleic acid molecule in a biological sample that contains many nucleic acid molecules that do not have a genetic variation in the region being scanned. A nucleic acid region being scanned may be at least about 10 bases long, for example about 20 bases, about 50 bases, about 100 bases, about 150 bases, or about 200 bases long. However, in some embodiments the region may be shorter, longer, or of intermediate length.

Methods of the invention can be used to screen for mutations that are predictive or indicative of a disease state. The presence of a mutant nucleic acid molecule in a biological sample may be indicative of a disease such as cancer or pre-cancer (e.g., colon cancer or adenoma). Methods according to the invention are more sensitive than current sequencing methods and can detect, in a scanning reaction, the presence of relatively low frequency mutations in a heterogeneous biological sample. According to aspects of the invention, an altered/mutant nucleic acid molecule originating from an adenoma and/or early stage cancer cell (or debris thereof) may be shed into a biological sample along with a large number of corresponding normal nucleic molecules that are shed from normal cells (i.e., non-adenoma and non-cancer cells) that line a lumen from which the biological sample originates or is obtained. An adenoma or early stage cancer is typically small and very few diseased cells (or debris thereof) are shed into the biological sample relative to normal cells (or debris thereof) from the normal tissue surrounding the adenoma or early stage cancer. As a result, altered/mutant nucleic acid molecules indicative of the adenoma or early stage cancer may be very rare relative to the corresponding normal nucleic acid molecules (i.e., nucleic acid molecules with an unaltered or non-mutant sequence from the same region of the genome as the altered/mutant nucleic acid molecule that has the altered/mutant sequence). According to aspects of the invention, indicia of certain later stage cancers also may be present at low frequencies in heterogeneous biological samples. Accordingly, aspects of the invention are useful for disease detection.

According to aspects of the invention, single base tracking reactions increase the sensitivity for detecting low frequency genetic events, at least because signals from bases at any one position in a sequence being scanned are no longer masked by signals from an alternate base in the wild type sequences present at higher concentrations in the sample. Sensitivity for low frequency mutations in a biological sample also may be increased by using certain ratios of extending nucleotides (nucleotides that can be extended in an enzymatic primer extension reaction) to terminating nucleotides; using two or more polymerases with different relative preferences for extending and terminating nucleotides; using certain analytical techniques (e.g., manual techniques, automated techniques, computer-implemented software, or any combination thereof) to quantify one or more signals associated with the incorporation of a known nucleotide (e.g., a labeled terminator nucleotide) at a known position in an extension reaction of the invention; or using any combination of the above techniques. Therefore, methods of the invention may be used to detect the presence of nucleotide sequences with altered residues when compared to a control “wild type” nucleotide sequence, where the nucleic acids with altered sequence make up about 50%, about 25%, about 10%, about 5%, about 4%, about 3%, about 2.5%, about 2%, about 1.5% or especially about 1% of the nucleic acids in the sample being analyzed, or even lower than 1%, for example about 0.5%, or about 0.1% or lower than 0.1% of the nucleic acids in the sample being analyzed.

In a preferred reaction, the terminator nucleotide is labeled. A preferred label is a fluorescent label, although it is within the skill of an artisan to use substitute labels of equal or higher sensitivity in signal detection, and/or equal or lower background signal noise. The DNA single base tracking reaction utilizes sensitive labeling techniques in order that the resulting sequence fragments may be analyzed and, e.g., compared to a known normal control sample to determine whether at least one genetic variation exists between the sample and normal control.

One aspect of the invention includes a method for detecting a difference between two nucleic acids. The method includes extending a first primer complementary to a target nucleic acid in the presence of a first nucleotide and a second nucleotide to produce at least one product. The first nucleotide is at least one deoxynucleotide, and more preferably is a mixture of four deoxynucleotides, namely dATP, dCTP, dGTP and dTTP (“dNTP mixture”) used for the elongation step of the primer extension reaction. The second nucleotide is a terminator nucleotide, preferably includes a detectable label, and has the same base as one of the first deoxynucleotides. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid. Signal differences include the addition of at least one peak, the deletion of at least one peak, or a shift in the position of at least one peak present in the sample as compared to the control.

In another aspect, a scanning reaction may be analyzed for signs of low frequency genetic events (e.g., one or more mutations) without using a comparison to a control or other reference nucleic acid of known sequence. For example, the presence of a mutation at a low frequency may be determined by quantifying the signals obtained for different positions of the primer extension product and determining whether one or more of the signals are present at a low, but statistically significant, level relative to signals for other positions (e.g., at about 10%, about 5%, about 1%, about 0.1% or lower than signals at other positions). Similarly, a corresponding loss of a small, but statistically significant, amount of signal (e.g., a loss of about 10%, about 5%, about 1%, about 0.1% or less than signals at other positions) at a position expected to generate a signal using a different terminator nucleotide may be indicative of (or confirm) the presence of a variant nucleotide at that position for a small number of nucleic acids in the sample being assayed.

The embodiments described above and below can have any or all of the following features. The method may include the step of amplifying a nucleic acid to form the target nucleic acid. The extending step can include extending the primer in the presence of the deoxynucleotides dATP, dCTP, dGTP, and dTTP. The target nucleic acid can be a nucleic acid suspected of containing a mutation. The target nucleotides to be screened in the methods of the invention may be genomic DNA, complementary DNA (cDNA), or RNA. Where the initial sample is RNA, it is preferred that the RNA is converted into DNA prior to further processing. The extending and comparing steps can be repeated. The extending and comparing steps can be conducted two or more times (e.g., at least four times) with the same primer, each time using a different one of adenine (A), cytosine (C), guanine (G) or thymidine (T) for the base of the second “terminating” nucleotide (i.e., each extension reaction contains only one type of extension terminating nucleotide, where the terminating nucleotide may be a dideoxynucleotide or an acyclonucleotide, and the base of the terminating nucleotide is chosen from A, C, G, or T).

A comparison nucleic acid can be a wild type nucleic acid. The signal from the comparison nucleic acid can be determined prior to, at the same time as, or after the signal from the target nucleic acid. The signal can include a fluorescent light emission. Alternatively, the signal results of the control sequence may be obtained from a database of nucleotide sequences. The comparison step may be done manually or by automation.

The methods described above or below can also have any or all of the following features. In certain embodiments, the method includes extending a second primer complementary to the target nucleic acid in the presence of the first nucleotide and the second nucleotide to produce at least one secondary product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one secondary product is the product of a primer extension reaction. The method may also include detecting a signal from the at least one secondary product and comparing the signal from the at least one secondary product with a signal that was generated from a comparison nucleic acid in substantially the same manner as the signal was generated from the target nucleic acid. A difference between the signals indicates at least one difference between the target nucleic acid and the comparison nucleic acid.

The methods described above or below may also include the following features. In one embodiment, a second primer complementary to a strand complementary to the target nucleic acid is extended in the presence of the first nucleotide and the second nucleotide to produce at least one secondary product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one secondary product is the product of a primer extension reaction. The method can then include detecting a signal from the at least one secondary product and comparing the signal from the at least one secondary product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide including a detectable label and a second nucleotide to produce at least one product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that is generated from a comparison nucleic acid in substantially the same manner as the signal is generated from the target nucleic acid. A difference between the signals indicates at least one difference between the target nucleic acid and the comparison nucleic acid.

In another aspect of the invention, a method for detecting a difference between two nucleic acids includes extending a first primer including a detectable label and being complementary to a target nucleic acid in the presence of a first nucleotide and a second nucleotide to produce at least one product. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a signal from the at least one product and comparing the signal from the at least one product with a signal that was generated from a comparison nucleic acid in substantially the same manner as the signal was generated from the target nucleic acid. A difference between the signals indicates at least one difference between the target nucleic acid and the comparison nucleic acid.

In another aspect of the invention, a method for detecting a difference between two nucleic acids includes extending a first primer complementary to a target nucleic acid in the presence of a first nucleotide and a second nucleotide to produce at least one product. The second nucleotide is a terminator nucleotide and includes the same base as the first nucleotide. In a preferred embodiment, the first nucleotide is a mixture of extending nucleotides (e.g., a deoxynucleotide (dNTP) mixture), the second nucleotide is a terminator nucleotide (dideoxynucleotide or acyclonucleotide) of only one base selected from A, C, G or T, and the at least one product is the product of a primer extension reaction. The method also includes detecting a mass of the at least one product and comparing the mass of the at least one product with a mass that is generated from a comparison nucleic acid in substantially the same manner as the mass is generated from the target nucleic acid. A difference between the masses indicates at least one difference between the target nucleic acid and the comparison nucleic acid. Product masses may be determined using electrophoresis, mass spectrometry, or any other suitable technique as the invention is not limited in this respect.

For any of the assays described herein, a single base-tracking primer extension reaction may be cycled 2 or more times, for example between 5 and 40 times, about 30 times (e.g., 28, 29, 30, 31, or 32 times). For any of the assays described herein, PCR primers of between 20 and 50 nucleotides in length may be used. Similarly, primers used in single-base tracking extension reactions may be between about 20 and 50 nucleotides (e.g., about 30 or about 40) in length. However, other numbers of reaction cycles and primer lengths (e.g., longer, shorter, or intermediate lengths) may be used as the invention is not limited in this respect. It should be appreciated that the number of reaction cycles and primer lengths may be adapted to enhance the signal to noise ratio for detecting low frequency mutations in single-base scanning reactions described herein. However, it also should be appreciated that if a positive result is obtained using a single-base scanning reaction, the same assay may be repeated and/or a further assay may be performed in order to confirm the presence of a mutation and/or the frequency of the mutation in the biological sample.

In one aspect, base-tracking reactions may be performed with a ratio of extending nucleotides to terminating nucleotides of about 50:1 (for example a ratio of about 12.5:1 for each individual extending nucleotide to the terminating nucleotide). However, lower ratios may be used provided that the rate of misincorporation of the labeled nucleotide does not exceed the level of mutant nucleic acid that is being assayed for. Ratios of extending nucleotides to terminating nucleotides may be between about 10:1 and about 100:1 (e.g., about 10:1, 20:1, 30:1, 40:1, 50:1, 60:1, 70:1, 80:1, 90:1, or an intermediate value). However, other ratios may be used as the invention is not limited in this respect. Typically, a ratio of extending nucleotides to terminating nucleotides may be lower than one used in a standard sequencing reaction but higher than one used in a standard primer extension reaction (e.g., single base extension reaction) designed to detect a specific mutation. A ratio may be chosen so that the single base-scanning reaction extend for between about 20 to about 150 bases, for example for between about 50 and about 100 bases so that any low frequency genetic event produces sufficient signal to be detected and/or quantified.

The products of the single base tracking reactions (e.g., from each amplification fragment) may be separated using electrophoresis, e.g., CAGE (capillary gel electrophoresis). The shorter fragments elute from the gel first while the longer fragments elute from the gel last. As the fragments are eluted from the gel, the signal from the label is detected and a pattern of signals for all the fragments is determined (examples of patterns are shown in the Examples below). For example, if fluorescent labels are used, an ABI 3100 DNA Sequencer can be used to read out the signal pattern. Each reaction can be analyzed independently and/or multiplexed.

In one embodiment, the same single base tracking procedure may be carried out for a known normal nucleic acid sequence (e.g., a wild type sequence). The signal pattern generated for the normal sequence may be compared with the signal pattern from the sample. Insertions, deletions and point mutations may be identified by a change in the peak pattern relative to the wild type peak pattern. This comparison can be undertaken manually or in an automated fashion. For example, comparisons may be performed using software that quantifies the amount of different types of signals at different positions (e.g., for extension products of different lengths). The amount of a signal at a position that is not expected to have a signal in a wild-type template may be used to determine a frequency of mutant or variant nucleic acids in a biological sample. This modified sequence reaction produces results that can detect a mutant to wild type ratio in a sample of less than 1:1. For example, the invention can detect mutations present at a ratio of about 1:4 (mutant to wild type), about 1:10 (mutant to wild type), about 1:100 (mutant to wild type), or less than about 1:100 (mutant to wild type), for example about 1:1,000 or less. Accordingly, this tracking method has a higher sensitivity for mutations than do current sequencing reactions.

Additionally, the single base tracking reactions described herein can be run in the forward and reverse directions. For example, if the procedure described above was the “forward” direction, primers would be designed to sequence the amplification fragments in the opposite direction. This reverse direction single base tracking is a manner to confirm that the result obtained in the forward direction is accurate. The single base tracking reaction can be run in the forward and/or reverse directions as many times as is desired to confirm the results.

In a first step, an embodiment of the invention may include preparing a nucleic acid sample (typically DNA) for scanning. In some embodiments, a nucleic acid preparation may be amplified from a biological sample. To the extent enough nucleic acid exists in the sample, amplification is not required. However, to the extent amplification is desired, any of a variety of methods can be used including, but not limited to PCR, RT-PCR, OLA, rolling circle, single base extension, and others such methods known to one skilled in the art. To the extent PCR is used, primers are designed to amplify a targeted region of a genome or other source of nucleic acid. For example, the region may be a mutation cluster region (“MCR”) or any other region suspected of being associated with a mutation diagnostic for a disease (e.g., mutations present in a gene such as APC, p53, BAT-26, PIK3CA, beta-catenin, or a portion thereof such as exon 9 or exon 20 of the PIK3CA locus, exon 5 of the beta-catenin locus, or a portion of any of the above suspected of containing a mutation). Nucleic acid regions that may contain one or more nucleic acid mutations associated with cancer are described for example in Cancer Research (Mar. 15, 1998) 58, pp 1130-1134, and Science (Apr. 23, 2004) 304(5670): 554, the disclosures of which are incorporated herein in their entirety. However, aspects of the invention may be useful for scanning any genomic region in which one or more mutations may be associated with diseases such as cancer (e.g., a “hotspot” region for mutations associated with diseases such as cancer). If using PCR, a primer pair may be designed to amplify the entire region in one reaction. Alternatively, several primers can be designed to overlap. In this case, two or more sets of primers are used to amplify the region. The sets of primers can be used in separate amplification reactions or in one multiplex reaction.

One of the PCR primers that generates each fragment may be biotinylated if post-PCR cleanup is desired. For example, the biotinylated PCR amplification fragments can be run over a column having complementary streptavidin bound to beads. This removes the amplified fragments from the rest of the nucleic acid in the amplification reactions, simplifying the ability to see relatively low amounts of mutant nucleic acid. The bound amplification fragments are then optionally eluted from the column. Alternatively, other binding partners that are known in the art may be used with one of the partners attached to the PCR primer. Attachment may be by any suitable linkage or linkage method known to one skilled in the art.

Target Molecules

It should be appreciated that in order to determine with statistical significance whether an abnormal nucleic acid is present in, or absent from, a biological sample, a minimum or threshold number of genome equivalents of a target nucleic acid need to be characterized (e.g., sequenced in whole or in part) to determine if any one of them is abnormal. For suspected rarer abnormalities, higher numbers of genome equivalents need to be characterized to reach a statistically significant conclusion that the sample does or does not contain the abnormality. For example, if a mutation is suspected to be present in 1% (if at all) of the copies of a target nucleic acid in a sample, then 100 or more copies (genome equivalents) of the region suspected to be mutant should be characterized. In this embodiment, the result has higher statistical significance if about 200; 300; 400; 500; 600; 700; 800; 900; 1,000 or more target nucleic acid molecules are sequenced. In one aspect, a statistically significant result may be obtained for an abnormality suspected to be present in x % of the target nucleic acids in a biological sample (or in x % of the captured nucleic acid molecules) if 100/x or more genome equivalents of a target nucleic acid containing the region suspected of being abnormal are characterized. In certain embodiments, about 200/x; about 300/x; about 400/x; about 500/x; about 600/x; about 700/x; about 800/x; about 900/x; about 1,000/x; about 5,000/x; about 10,000/x; about 50,000/x; about 100,000/x; about 500,000/x; about 1,000,000/x or more genome equivalents are isolated, captured, analyzed, and/or characterized. For example, if a 0.1% level of abnormality is suspected, 1,000 or more genome equivalents should be characterized. Similarly, for a 0.01% level, 10,000 or more genome equivalents should be characterized. Accordingly, appropriate sample volumes and isolation steps should be used to provide sufficient genome equivalents for subsequent analysis. It should be appreciated that less than 100/x genome equivalents may be used under certain circumstances where statistical significance is less important.

In certain embodiments, two or more markers may be analyzed in a single assay. Accordingly, two or more different target nucleic acid regions may be isolated. In one embodiment, the number of genome equivalents of each target molecule is above a threshold number sufficient for a statistically significant result to be obtained upon subsequent sequence analysis of the captured molecules or a portion thereof. In general, the threshold level would be set at the same level for each different abnormality being assayed for in a biological sample.

It should be appreciated that the level of sensitivity (e.g., how low a percentage of abnormality can be detected) may determine the earliest stage at which the presence (e.g., recurrence) or absence of a disease may be detected with statistical significance. For example, if a predetermined threshold level of at least 10,000 genome equivalents are characterized, a 0.01% level of mutation may be detected with statistical significance. Detecting a mutation at a 0.01% level allows a disease to be detected earlier than using a 0.1%, 1%, 10% detection level, because the 0.01% level corresponds to a stage in the disease when the diseased cells have not multiplied to a level that would allow them to be detected using a 0.1%, 1%, or 10% detection threshold. Similarly, a 0.1% threshold allows earlier detection than a 1% threshold (and 1% earlier than 10% etc.). Characterizing hundreds or thousands of (e.g., 5,000; 10,000; 50,000; 100,000 or more) copies of a single genetic region or of each of several genetic regions may seem like a large amount of work. However, high complexity analytical methods such as those developed for genome sequencing (and particularly those developed for single molecule sequencing) can be used for this task since they are capable of sequencing many more molecules than required for statistical significance according to the invention. For example, the number of single molecules required for sequencing an entire genome, or even a significant portion of a genome, is greater than the number of single molecules that may be sequenced for statistical significance according to certain aspects of the invention. A particular feature of methods of the invention is that the single molecules being sequenced have similar, identical, or overlapping sequences, because they were isolated as multiple genome equivalents including a locus of interest. This differs from genome sequencing where most of the single molecules being sequenced have different sequences since they are generated to represent different portions of the genome. Accordingly, while methods of the invention use high-complexity analytical techniques, these techniques may be adapted for the particular configurations required by aspects of the invention. For example, a predetermined genetic locus may be analyzed using a single sequencing primer that is expected to work on all of the isolated target molecules. This primer may be sequence specific and contain at least about 10, 15, 20, 25, 30, 35, 40, 45, 50, or more nucleotides that are complementary to a region of the target molecule in proximity with the region suspected of containing an abnormality. In some embodiments, two or more different primers (e.g., 3, 4, 5, 6, 7, 8, 9, 10, etc.) may be used in different sequencing reactions (for example, each using a threshold number of genome equivalents of target nucleic acid) to provide further confidence in the sequencing results. In contrast, certain genome sequencing methods involve a plurality of different primers in a single analysis so that each primer can hybridize to, and provide sequence information for, a different part of the genome. Similarly, data or sequence analysis techniques of the invention may be adapted for comparing many copies of a similar, identical, or overlapping sequence in order to determine if one or more of the molecules being characterized (e.g., sequenced) contains a genetic abnormality of interest. It should be appreciated that a genetic abnormality may be any form of mutation (for example, a point mutation, a transition, a transversion, a duplication, a deletion, an inversion, a translocation, or any other form of mutation). In certain aspects of the invention, analytical methods also may be adapted to detect rare modified nucleic acids such as hyper- or hypo-methylated nucleic acids that may be associated with a disease.

Aspects of the invention may involve using a high number of amplification cycles to reach a point at which amplification is saturated in order increase the probability of amplifying any mutant templates that are present in a sample. For example, the number of amplification reactions may be above 30, for example above 40, above 50, about 60 or above. In one embodiment, an entire amplification product may be analyzed (e.g., in one single base scanning reaction). In other embodiments, an entire amplification reaction may be partitioned into aliquots that are analyzed using two or more different assays (e.g., single base scanning reactions), optionally with two or more primers.

However, in some embodiments, due to the stochastic nature of nucleic acid recovery in typical sample preparations and the stochastic nature of nucleic acid amplification reactions, the presence of one or a small number of abnormal nucleic acids may not be detected even if a large number of amplification cycles are used. For example, normal nucleic acids may be “preferentially” amplified in the first few rounds of an amplification reaction even if abnormal nucleic acids are present in the sample. This may occur due to the stochastic nature of the amplification reaction (for example, not all templates are amplified in each cycle) and the presence of only very small numbers of abnormal nucleic acid molecules in the biological sample. This “preferential” amplification of normal nucleic acid molecules in the first few cycles may result in an over-representation of the normal nucleic acid in the final amplified product (and therefore a lower percentage of abnormal nucleic acid in the final amplified product than in the initial biological sample). Therefore, in one aspect, methods of the invention may include a high yield or high efficiency nucleic acid preparation and/or capture procedure in order to isolate as much abnormal nucleic acid as possible from a biological sample.

In some embodiments, a predetermined (e.g., theoretically expected or calculated, or experimentally determined) number of genome equivalents may be helpful to obtain a predetermined level of detection sensitivity. For example, statistics (e.g., based on an analysis of a Poisson distribution) may predict that about 500 genome equivalents of a predetermined genetic locus may be required in order to obtain a 99% probability of amplifying a nucleic acid molecule (e.g., a mutant DNA molecule) that represents about 1% of the copies of the genetic locue in the sample. Similarly, in some embodiments about 2500 genome equivalents may be required in order to obtain a 99% probability of amplifying a nucleic acid molecule that represent only 0.2% of the sample. Accordingly, in some embodiments at least 500/x genome equivalents may be required for a molecule that represents x % of the copies of the molecule in a sample. In some embodiments, x may represent a threshold detection level. For example, x may be set at 1, 0.1, 0.01 or other suitable level so that the assay is designed to detect abnormal nucleic acids that are present at least at 1%, 0.1%, 0.01% levels, respectively, in a heterogeneous biological sample. As described herein, in some embodiments, due to the stochastic nature of nucleic acid retrieval and/or amplification, higher than a theoretically predicted threshold number of genome equivalents may be used (e.g., 1.5 fold, 2 fold, 3 fold, 4 fold, 5 fold, 5-10 fold, 10 fold or more that theoretically determined). For example, a higher than expected or calculated number of genome equivalents may be used. For example at least 600/x, at least 700/x, at least 800/x, at least 900/x, at least 1000/x, at least 1250/x, at least 1500/x, at least 1750/x, at least 2000/x, at least 2500/x, at least 3000/x, at least 4000/x, at least 5000/x, at least 10000/x, or more genome equivalents may be used. It should be appreciated that when screening a population of individuals it may be important to use a retrieval technique that reliably captures at least 500/x genome equivalents and preferably more than that (e.g., at least 600/x or one of the other higher numbers of genome equivalents as described herein). Certain assays may be more susceptible to increased sensitivity with an increase in the number of genome equivalents. For example, certain assays that involve detecting an amount (rather than presence or absence) of a certain nucleic acid may be more susceptible to increased signal using higher genome equivalents. For example DIA assays may be particularly susceptible to increased sensitivity when using higher than an expected threshold number of genome equivalents. Also, methylation detection assays (e.g., methylation specific amplification assays using methylation specific primers, and e.g., using a methylation specific modification agent such as bisulfite) may be more sensitive if more than a theoretically predicted threshold level of genome equivalents are used. However, as described herein, other assay formats (e.g., primer extension based assays, hybridization assays, an/or other assays described herein) also may benefit from using higher starting genome equivalents in order to provide a robust assay that reliably detects mutations if they are present in a patient sample (e.g., with a clinical sensitivity of at least 60%, 70%, 80%, 90%, 95%, 99%, or more etc.). Increased genome equivalents may be particularly important for any detection or characterization assay that uses an amplified nucleic acid sample for analysis. Accordingly, in some embodiments an assay may involve recovering a high number of genome equivalents of a predetermined locus as described herein, performing an amplification reaction (e.g., PCR), and analyzing the amplified product. However, in other embodiments, digital analyses (e.g., single molecule detection and/or sequencing assays) and/or other detection methods may benefit from higher genome equivalents in order to provide an assay format that reliably detects mutations if they are present in a patient sample. Accordingly, in one embodiment, an assay format that is suitable for screening individuals with no known indicia of disease yields improved and more robust results if at least 500/x genome equivalents (and preferably higher number, e.g., 600/x and other higher numbers described herein) of a predetermined locus are recovered and used for analysis. In one embodiment, a patient population may be screened by maximizing the number of patient samples that include at least 500/x genome equivalents (and preferably higher number, e.g., 600/x and other higher numbers described herein). An individual patient assay may be determined to be less reliable when the tested number of genome equivalents is identified and determined to be below a predetermined threshold level as described herein. Accordingly, based on the number of genome equivalents that are tested a confidence level of the results may be evaluated. In some embodiments, if a patient sample contains less than a predetermined level of genome equivalents then the results may be marked to indicate a lower confidence level than that of other samples. Alternatively, a patient may be asked to provide an additional sample for processing (or additional sample may be processed if the patient already had provided additional sample). It should be appreciated that high sample volumes and/or high efficiency capture techniques may be useful in a population screen, particularly since typical healthy patients often yield samples with less nucleic acid than diseased patients. Even if a patient sample contains low amounts of DNA, this alone may not be sufficient to determine that the patient is healthy. Preferably, an assay on at least a threshold number of genome equivalents is performed. Even for samples with low amount of nucleic acid, retrieving and assaying higher numbers of genome equivalents provides a higher confidence level that a negative result (absence of indicia of disease) is reliable or significant. It should be appreciated that these considerations apply to all samples described herein. Methods for obtaining predetermined genome equivalents are described herein and may involve using a sequence specific capture probe attached to a solid support. Amplification reactions may involve sequence specific primers as described herein.

High Efficiency Isolation of Target Molecules

Any method that is suitable for isolating a threshold number of genome equivalents of one or more target molecules may be used in certain aspects of the invention. In preferred embodiments, a specific hybrid capture method may be used. A hybrid capture method may involve using a capture probe to bind to a target nucleic acid. The bound product then may be isolated. In one embodiment, a capture probe may be bound to a solid surface thereby acting as an anchor for isolating a target molecule. In other embodiments, a capture probe may be modified in a manner that allows it to be isolated or purified from a sample. For example, a capture probe may biotinylated, attached to an antigen, attached to a magnetic particle, attached to a molecular weight marker, attached to a charged particle, attached to another particle or other molecular “hook” that can be used to isolate that capture probe and thereby isolate a target molecule that is hybridized to the probe. In some embodiments, a capture probe binds to (and captures) a target molecule at a region near a region suspected of containing an abnormal sequence. Accordingly, a capture probe may bind to a normal sequence and capture target molecules with or without a genetic abnormality. However, in some embodiments a capture probe may be specific for a predetermined mutation and preferentially bind to target nucleic acids containing the mutation.

In aspects of the invention, a nucleic acid preparation is captured by repeated exposure of a biological sample (for example, a processed biological sample) to a capture probe on a solid support or in a medium, for example, by the rapid flow of the sample past a capture probe for the target nucleic acid molecule. The repetitive nature of such a method allows for a target molecule to bind and enhances the total number of molecules bound to the capture probe, providing a high yield capture. The solid support may be an electrophoretic medium (e.g., gel or beads) and the repetitive exposure of the sample to the capture probe may involve exposure to repeated cycles of electrophoresis in alternate directions (back and forth across a solid support region containing one or more different types of capture probe). In some aspects, a sample is added to a portion of an electrophoretic medium having at least two regions arranged consecutively in a first spatial dimension. In some aspects, at least one of the at least two regions includes a first capture probe which is immobilized within that region. An electric field is applied to the electrophoretic medium in a first direction which is parallel to the first dimension. An electric field is then applied to the electrophoretic medium in a second direction which is opposite to the first direction. In further aspects, the electric field is applied repeatedly in each direction. For further details see for example U.S. Application No. 60/517,623 and U.S. application Ser. No. 10/982,733, the entire contents of which are incorporated herein by reference.

In aspects of the invention, a sample may be exposed repeatedly to a capture probe using chromatographic methods, for example high performance liquid chromatography (HPLC), fast performance liquid chromatography (FPLC), etc.

In some embodiments, the captured sample may be amplified using PCR (or other amplification technique) to obtain a pool of DNA of an expected size. However, amplification is not required as the invention is not limited in this respect.

In some aspects of the invention, a capture probe may be any molecule capable of binding a target molecule (or a non-target molecule as described below). According to the invention, a target molecule is a molecule that contains a region suspected of being altered or mutant in disease (e.g., in adenomas or early stage cancers). Accordingly, a capture probe binds to a portion of a nucleic acid that is adjacent to (or overlaps) a position or region suspected of being mutant or altered. The capture probe should bind sufficiently close to the suspected position or region to effectively capture a significant number of target molecules that contain the suspected position or region. For example, in one embodiment the capture probe should bind to a portion of a nucleic acid that is within 5,000 bases (e.g., within 2,500 bases, within 1,000 bases, within 750 bases, within 500 bases, within 250 bases, or within 100 bases) of the position or region suspected of being mutated or altered. A capture probe may be between about 30 and about 40 bases long (e.g., about 31, 32, 33, 34, 35, 36, 37, 38, or 39 bases long). However, shorter or longer capture probes may be used. In some aspects, a capture probe selectively binds to a target molecule in a sample. In one embodiment, a capture probe is outside of a region of the nucleic acid to be amplified. It should be appreciated that a capture probe may bind to target nucleic acid molecules with overlapping sequences, because nucleic acid fragmentation (e.g., resulting from natural fragmentation or exposure to a fragmentation technique) typically generates overlapping fragments of different sizes.

According to aspects of the invention, the capture probe can bind a target molecule during electrophoresis under appropriate conditions, such as pH, temperature, solvent, ionic strength, electric field strength etc. One of ordinary skill in the art would be able to adjust any condition as required to achieve optimal binding. A capture probe may include, but is not limited to, one or more peptides, proteins, nucleic acids, amino acids, nucleosides, antibodies, antibody fragments, antibody ligands, aptamers, peptide nucleic acids, small organic molecules, lipids, hormones, drugs, enzymes, enzyme substrates, enzyme inhibitors, coenzmyes, inorganic molecules, polysaccharides, and/or monosaccharides.

When a nucleic acid capture probe is used (e.g., an oligonucleotide, a DNA, an RNA, a PNA, or other form of natural, synthetic, or modified nucleic acid) it should have a sequence that is sufficiently complementary to a portion of the target nucleic acid to bind specifically to the target nucleic acid under the conditions used for capture. In some embodiments, the capture probe may have a sequence that is 100% complementary. However, in other embodiments, the sequence may contain a few non-complementary nucleotides (e.g., at the 3′ or 5′ end). It should be appreciated that a small number of non-complementary nucleotides may be non-complementary. For example, the capture probe may be between 80% and 100% complementary (e.g., about 85%, about 90%, or about 95% complementary) to a portion of the target nucleic acid. However, other degrees of complementarity may be used provided that the capture probes can capture a sufficient number of genome equivalents of a target nucleic acid with sufficient specificity for subsequent analysis. It should be appreciated that aspects of the invention do not require a pure sample of target nucleic acids. Nucleic acids other than the target nucleic acids may be isolated and included in the analytical step provided that they do not interfere with the sequence analysis in a way that would reduce the significance of the results to a level that falls below a predetermined level of statistical significance.

In embodiments of the invention, exposure of a biological sample (for example a crude preparation of total nucleic acid from a biological sample) to immobilized capture probe(s) may be repeated between 2 and 100 times, e.g., between about 5 and about 50 times, between about 10 and about 40 times, or about 11, 12, 13, 14, 15, 16, 17, 18, 19, 20, etc. times, including about 25, 30, or 35 times.

A captured preparation of target nucleic acid molecules (e.g., of low genomic complexity) may be eluted using any suitable technique and prepared (e.g., single stranded molecules may be prepared) for subsequent analysis using a technique for analyzing nucleic acid samples of high genomic complexity. Sample capture techniques described herein may be used to analyze DNA and/or RNA.

Colonic Samples

In aspects of the invention, an increase in sensitivity may be achieved by using large amounts of sample. In embodiments of the invention, a large amount of sample may be processed in order to increase the confidence level of isolating or capturing a rare event indicative of very early stage disease (e.g., an adenoma, an early stage cancer, recurrence, etc.). In some embodiments, large volumes of sample may be processed in order to obtain sufficient genome equivalents of one or more target nucleic acids of interest (e.g., target nucleic acids that are being screened for the presence of a mutation, or target nucleic acid(s) that are being monitored for the progression or recurrence of a genetic abnormality that has been associated with a disease in a patient). For example, about 5 g, about 10 g, about 15 g, about 20 g, about 25 g, about 30 g, about 35 g, about 40 g, about 45 g, about 50 g, about 55 g, about 60 g, about 65 g, about 70 g, about 75 g, about 80 g, about 85 g, about 90 g, about 95 g, about 100 g, about 150 g, about 200 g, or more sample (e.g., stool sample) may be processed using a capture technique described herein. For example, more than about 5 mls, about 10 mls, about 15 mls, about 20 mls, about 25 mls, about 50 mls, about 75 mls, about 100 mls, about 200 mls, about 300 mls, about 400 mls, about 500 mls, about 1 liter, or more colonic effluent may be analyzed or processed to isolate nucleic acid for subsequent analysis. Intermediate volume ranges between any of the specific volumes set forth above also may be used.

In one embodiment, aspects of the invention involve screening isolated nucleic acids for mutations, hypermethylation, and/or integrity. The isolated nucleic acids may be cell free and may be isolated from colonic effluent. In one embodiment, an important step in the isolation of cell-free nucleic acids from colonic effluent may be a filtration step. For example, the filtration may involve passing a sample of colonic effluent through a filter that excludes cells but not free nucleic acids (e.g., a 0.45 micron filter or smaller).

Nucleic Acid Preservation:

Methods for Stabilizing Nucleic Acids in Biological Samples

According to aspects of the invention, stabilization solutions may be particularly useful for preserving nucleic acids to detect one or more indicia of disease.

In certain embodiments, high sensitivity may be achieved by preserving nucleic acid integrity in colonic samples. It has been unexpectedly found that an abnormal DNA molecule can be stabilized and/or enhanced by mixing, or incubating, a patient sample known to or suspected of containing DNA indicative of a disease with a stabilization solution prior to performing a DNA integrity assay or other nucleic acid assay described herein. The stabilization solution typically includes one or more buffers, and/or chelating agents, and/or salts. Aspects of the invention are particularly useful for nucleic acid integrity assays. However, mutation detection (for example, in a multiple mutation assay) and hypermethylation analysis also can benefit from stabilization.

According to the invention, an important challenge for cancer (e.g., colon cancer) detection from stool is to preserve the integrity of human DNA in the hostile stool environment, in order to recover, amplify, and interrogate the DNA for known cancer related abnormalities. Nucleases that are active in stool have the potential to rapidly degrade DNA, including the minor human DNA component, and measures may be taken to minimize their negative impact. Typically, clinical samples are frozen as quickly as possible after collection. However, in order to use fecal DNA tests in population screens, it should be expected that there will be some variability in the time between sample collection and shipping to testing labs, and furthermore, some variability in the temperature at which stool samples are transported. In order to eliminate any variables in sample handling that might have an impact on assay performance we have run controlled sample incubation experiments and looked at how different markers in a multi-target assay are affected. Similar considerations also may apply to colonic effluent samples.

Markers may be chosen that yield an acceptable clinical sensitivity for the intended application such as screening a population for indicia of a disease. In addition, for stool sample analysis, mutation detection methods should offer sufficient analytical sensitivity since the human DNA recovered from stool is highly heterogeneous. Normal cells are sloughed into the colonic lumen along with the mutant cells. Therefore, in one embodiment, analytical methods should detect as little as 1% (or less) mutant DNA in the presence of excess wild-type DNA. Also, certain sample preparation methodologies may be used for maximum recovery of human DNA from samples. The vast majority of DNA recovered from stool often is bacterial in origin, with the human DNA component representing only a small minority. Certain purification methodologies can efficiently select for the rare human component, and since the mutant copies (when they exist) represent only a small percentage of the total human DNA from stool it may be important to maximize the recovery of human DNA in order to maximize the probability of amplifying mutant copies in the PCR reactions. In one embodiment, gel electrophoresis methods for capturing human DNA may be used. However, according to the invention, it may be particularly important to preserve sample DNA for purification, especially when looking for early indicia of diseases (e.g., indicia of adenomas or early stage cancers that may be present in less than about 1%, or about 0.1% or less of human genomes isolated from a stool sample). A common method to insure that DNA remains stable is to freeze samples as quickly as possible after collection, or to receive samples in centralized testing labs as quickly as possible. However, in order to provide the option of decentralized sample analysis and still retain maximum sample integrity, it is desirable to use a more robust and standardized sample handling method. Similar considerations may apply for handling colonic effluent samples. However, in some embodiments, a sample may be analyzed immediately after retrieval (e.g. at the same time or immediately after the virtual imaging procedure).

In one aspect, the invention provides methods for stabilizing colonic samples by adding a stabilization solution to a sample as soon as possible after the sample is obtained. Methods of the invention do not require refrigeration or freezing. Aspects of the invention are based, in part, on the surprising discovery that nucleic acids in certain biological samples are stable at room temperature for hours, and even days (e.g., 1 day, 2 days, 3 days, or longer). However, in certain embodiments, samples with stabilization solution may be refrigerated or frozen. Aspects of the invention are particularly useful for preserving samples for nucleic acid integrity analysis. However, methods of the invention may be used to preserve samples for other assays including mutation detection and/or hypermethylation assays. In certain embodiments, methods of the invention are used to preserve a sample for analysis using a nucleic acid integrity assay along with a mutation detection assay (e.g., a multiple mutation panel assay), a hypermethylation assay, or any combination thereof.

Nucleic acid integrity assays are known in the art and are described in, e.g., US Patent Application No. 20040043467, US Patent Application No. 20040014104, U.S. Pat. No. 6,143,529, and Boynton et al., Clin. Chem. 49:1058-65, 2003. Nucleic acid integrity assays are based on higher levels of intact nucleic acid that appear in debris from cells that lyse non-apoptotically. Healthy patient generally produces cellular debris through normal apoptotic degradation, resulting in relatively small fragments of cellular components in tissue and body fluid samples, especially luminal samples. Patients having a disease generally produce cells and cellular debris, a proportion of which has avoided normal cell cycle regulation, resulting in relatively large cellular components. As a result, the disease status of a patient is determined by analysis of patient cellular components produced in specimens obtained from the patient. The presence of such fragments is a general diagnostic screen for disease.

Nucleic acids in patient samples tend to degrade after they have been removed from the patient. This degradation can diminish the effectiveness of a nucleic acid integrity assay that scores a sample as diseased (e.g., cancerous) based on the presence of intact nucleic acids; if the sample is excessively degraded, a sample that is actually positive may appear to be negative. While not wishing to be bound by theory, it is postulated that the stabilization buffer of the invention inhibits the nucleases that degrade the nucleic acids present in the diseased patient samples.

In some aspects of the invention, the addition of a stabilization solution to a biological sample may be used to preserve nucleic acid molecules containing one or more mutations that may be detected in a multiple mutation analysis (e.g., an analysis that involves interrogating a sample for the presence of a mutation at one or more loci, for example at about 2, about 3, about 4, about 5, about 10, about 15, about 20, about 25, or more loci). In some embodiments, the addition of a stabilization solution may be used to preserve nucleic acid for a methylation specific analysis to detect the presence of hyper-methylated nucleic acid molecules at one or more loci that may be indicative of cancer, adenoma, or other disease. In some embodiments, the addition of a stabilization solution may be used to preserve nucleic acid for a combination of a nucleic acid integrity assay and/or a multiple mutation analysis and/or a methylation detection assay. Assays may be performed under conditions to detect a small amount of mutant nucleic acid in a heterogeneous sample containing an excess of non-mutant nucleic acid (e.g., where the mutant nucleic acid represents less than 10%, less than 5%, less than 1%, or about 0.1% or less of the nucleic acid at a particular locus). In some embodiments, a digital assay may be performed on the preserved nucleic acid in order to detect rare genetic events. In some aspects, stabilization methods of the invention may preserve more than 80%, 85%, 90%, 91%, 92%, 93%, 94%, 95%, 96%, 97%, 98%, 99% or more of the nucleic acids indicative of a disease (e.g., long nucleic acid fragments, nucleic acid molecules containing one or more specific mutations, and/or hyper-methylated nucleic acid molecules).

The stabilization solution can be applied to a biological sample that is isolated directly from a patient, i.e., a freshly isolated biological sample. Alternatively, the method can be used on a biological sample that has been frozen (e.g., at −20° C. or −80° C.).

In aspects of the invention, stabilization solution may be added to a biological sample at any suitable ratio of sample to buffer. Ratios may be determined as a weight to volume (w/v) ratio. In some embodiments, the ratio may be about 1:1, about 1:2, about 1:3, about 1:4, about 1:5, about 1:6, about 1:7, about 1:8, about 1:9 or about 1:10 (w/v) sample to stabilization solution. However, higher or lower ratios may be used.

In aspects of the invention, the weight or volume of a biological sample may be determined before a buffer is added. According to the invention, it may be particularly important to immediately stabilize biological samples with a stabilization solution when the samples are to be interrogated for indicia of adenoma or early stage cancer. However, it also may be useful to stabilize biological samples for detecting indicia of later stage diseases or for monitoring disease progression.

In general, a stabilization solution may include one or more buffers and/or one or more chelating agents and/or one or more salts, or any combination of two or more thereof. The choices of buffer, chelating agent, and salt can be determined by the artisan. The suitability of a particular stabilization solution can be determined by comparing a nucleic acid integrity assay on samples that have been incubated with the stabilization solution to a parallel biological sample that has not been incubated with the stabilization solution. A suitable stabilization solution is a solution that shows a significant average fold increase in genome equivalents (GE) in a nucleic acid integrity assay compared to a GE determination made on parallel samples that have not been treated with the stabilization solution. Methods of calculating genome equivalents (GE) are known in the art (see, e.g., e.g., US Patent Application No. 20040043467, US Patent Application No. 20040014104, U.S. Pat. No. 6,143,529, and Boynton et al., Clin. Chem. 49:1058-65, 2003).

The temperature and pH optimum can also be determined empirically and optimized according to the combination of buffer, chelating agent and salt in the stabilization solution. While room temperature has been found to be a suitable temperature for incubating the patient sample and the stabilization solution, higher or lower temperatures (e.g., 4° C. to 16° C. or 25° C. to about 37° C.) can also be used, provided they do not undermine the effectiveness of the stabilization solution. The mixed patient sample and stabilization solution is preferably subjected to a minimum of agitation. However, according to the invention, the addition of a stabilization solution with little or no agitation is surprisingly effective at preserving nucleic acids for subsequent analysis.

In one aspect of the invention, a stabilization solution may be particularly useful when samples are not refrigerated or frozen or when there is a risk that a sample may not be maintained at a sufficiently low temperature to preserve indicia of disease. For example, a stabilization solution may be particularly useful if a sample is obtained at a remote location and mailed or delivered to a testing center. However, stabilization solutions also may be useful to preserve samples that are being processed on-site at a medical center.

Buffers

Suitable buffers may include, e.g. tris(hydroxymethyl) aminomethane, sodium phosphate, sodium acetate, MOPS, and other buffering agents as long as a buffer has the capacity to resist a 0.1 to 1 molar tris(hydroxymethyl)aminomethane or 0.1 to 1 molar phosphate ion. A combination of buffering agents can be used, so long as the solution has the required buffering capacity. Methods for determining the buffering capacity of a solution are well known in the art.

The comparison of buffering capacity is preferably carried out in the presence of the salt and chelating agent to be used in the stabilization solution, at the salt concentration to be used, and with the solutions being compared at about the same temperature, preferably at a temperature within the range of about 15° C. to about 25° C. In certain embodiments, high buffer concentrations may be used (e.g., higher than 50 mM, higher than 100 mM, higher than 200 mM, higher than 300 mM, higher than 400 mM, about 500 mM, or higher).

Chelating Agents

Chelating agents may be used in a stabilization solution. In some embodiments, chelating agents may be those that bind trace metal ions with high affinity. Non-limiting examples of chelating agents include, but are not limited to different forms of EDTA, EGTA, and other chelating agents. In certain embodiments, high chelating agent concentrations may be used (e.g., higher than 50 mM, higher than 100 mM, higher than 200 mM, higher than 300 mM, higher than 400 mM, about 500 mM, or higher). For example a stabilization solution may have an EDTA concentration of about 100 mM, about 150 mM, about 200 mM, about 250 mM, about 300 mM, or higher.

Salts

Candidate salts include, e.g., Nal, NaBr, NaCl, LiCl, KCI, KI, KBr, CsCl, GNHCl and GNSCN. In some embodiments, the salt is chaotropic and has an anion such as perchlorate, iodide, thiocyanate, acetate, trichloroacetate, hexafluorosilicate, tetrafluoroborate and the like. Cations for a chaotropic salt can include, e.g., the elements lithium, sodium, potassium, cesium, rubidium, guanidine and the like. More than one salt can be present in the buffered aqueous salt solution.

Similar stabilization methods may be used with different samples (colonic effluent or stool sample). Any of the buffers described herein may be added as soon as the sample is obtained (e.g., as soon as a stool sample is deposited or a colonic effluent is obtained).

In general, the stabilization solution is added to the patient sample at a ratio of about 1 ml/gram (or ml) of patient sample to about 20 ml/gram (or ml) of patient sample. In some embodiments, the stabilization solution is provided at 1-15 ml/gram (or ml), 2-12 ml/gram (or ml), 3-11 ml/gram (or ml), or 4-7 ml/gram (or ml). However, higher or lower ratios may be used. For example, a suitable ratio of stabilization solution to patient sample may be 7 ml/gm (or ml).

In some embodiments, the patient sample and stabilization solution may be incubated at about 4 to 28° C. In some embodiments the temperature is 17 to 27° C., e.g., about 20 to 25° C. However, the sample and stabilization solution may be exposed to higher or lower temperatures (e.g., the sample and stabilization solution may be frozen). Also, a sample and buffer may be exposed to changing temperatures during transport and/or storage.

Target Genes and Loci:

Genetic Loci and Genetic Abnormalities Associated with Disease

Aspects of the invention may be used to detect the presence of a genetic abnormality in any one or more loci of interest that may be associated with a disease. For example, one or more different loci associated with an adenoma, a cancer, a precancer or any other disease or disorder may be assayed according to methods of the invention. Examples of target nucleic acids include, but are not limited to, one or more oncogenes, tumor suppressor genes, genomic regions containing nucleic acid repeats (e.g., different forms of satellite DNA such as micro or mini satellite DNA), other genetic loci (coding or non-coding genetic loci), or combinations thereof.

In certain embodiments of the invention the presence or absence of mutations can be indicative of risk associated with developing cancer, early detection of cancer, and finally prognosis and treatment of cancer. Aspects of the invention may include detecting one or more mutations in one or more of the following genes: MSH2, MSH6, MLH1, PMS2, BUB1, BUBR1, MRE11, CDC4, APC, beta-catenin, TGF-beta, SMAD4, p21Waf1, 14-3-3sigma, PUMA, BAX, PRL-3, and PIK3CA. It is appreciated that these genes are involved at different stages of the neoplastic process and therefore can be selectively used to detect or monitor tumor origination, progression or recurrence. For example, mutations in the APC/beta-catenin pathway initiate the neoplastic process. Detection of mutations in these genes is very useful for cancer risk assessment. A patient with identified mutations in the APC/beta-catenin genes is at an elevated risk for developing tumors. Most often, mutations in the APC/beta-catenin genes result in adenomas (small benign tumors). These tumor progress, becoming larger and more dangerous, as mutations in other growth-controlling pathway genes accumulate. Growth-controlling pathway genes include K-Ras, B-RAF, PIK3CA, or p53. Detection of mutations in these genes could lead to early detection of the development of tumors. If undetected and untreated (in some circumstances) the neoplastic process can be accelerated by mutations in stability genes, such as PIKSCA/PTEN, PUMA, p53/BAX, p21Waf1, 14-3-3sigma, or PRL-3. Many of these gene products function to block cell birth, cell cycle and/or to activate cell death and apoptosis. Others like PRL-3 and PIK3CA are involved in regulating metastasis. Detection of mutations in these genes is significant for determining the clinical prognosis of a particular cancer, the treatment efficacy of a particular cancer treatment and for monitoring the progression or recurrence of a given tumor.

Adenomas

In one embodiment, aspects of the invention may be used to detect indicia of adenomas in a colonic sample. According to aspects of the invention, detecting the presence of an adenoma may be useful for detecting early signs of cancer or precancer. Adenomas are typically glandular tumors or tumors of glandular origin. Adenomas may be early indicia of cancer, for example colon cancer. Not all adenomas become cancers. However, many cancers (e.g., carcinomas such as colorectal carcinomas) are thought to develop from adenomas. Indeed, a majority of colon cancers are thought to develop from adenomas. Therefore, detecting adenomas is particularly useful for identifying early signs or risks of colorectal cancer (e.g., cancerous and precancerous lesions or growths in the colon).

Adenomas may be invasive adenocarcinomas, significant adenomas, and low potential polyps. Invasive adenocarcinomas may be, for example, adenocarcinomas at different TNM stages (e.g., TNM stages 1, 2, 3, or 4). Significant adenomas may be, for example, carcinomas in-situ/high-grade dysplasias (CIS/HGD) having a diameter of greater than 1 cm, about 1 cm, less than 1 cm, or of unknown size; villous adenomas having a diameter of greater than 1 cm, about 1 cm, less than 1 cm, or of unknown size; tubulovillous adenomas having a diameter of greater than 1 cm, about 1 cm, less than 1 cm, or of unknown size, and low-grade dysplasias (LGD) with a diameter of greater than or equal to 1 cm. Low potential polyps may be, for example advanced polyps, and adenoma low-grade dysplasias (LGD) with an unknown diameter or a diameter of less than 1 cm.

According to aspects of the invention, adenomas can be detected at different positions in the colon and rectum (including the right and left colon and the transverse colon).

In one embodiment, the following panel of genetic loci may be used to detect adenomas with greater than 60% sensitivity: assays may be performed to detect one or more genetic abnormalities from a multiple mutation panel of genetic abnormalities at 22 loci including KRas mutations in codon 12 (K12p.1, K12p.2) and codon 13 (K13p.2); mutations in APC codons 1309 (deletions), 1306 (mutations at position 1), 1312 (mutations at position 1), 1367 (mutations at position 1), 1378 (mutations at position 1), 1379 (mutations at position 1), 1450 (mutations at position 1), 1465 (deletions), 876 (mutations at position 1) and 1554 (insertions); mutations in p53 codons 175p.2, 245p.1, 245p.2, 248p.1, 248p.2, 273p.1, 273p.2 and 282p.1; and deletions at the BAT-26 locus. Mutations at these loci can be detected using primer extension assays (including single base extension assays and assays designed to detect micro-satellite instability such as BAT-26 deletions) or other assays that are useful to detect one or more of these genetic abnormalities.

In another embodiment, the following panel may be used to detect adenomas with greater than 60% sensitivity: assays are performed to detect hypermethylation at one or both of the HLTF and V29 loci. Hypermethylation at these loci can be detected using methylation specific primer analysis (e.g., MSP amplification) or other assays that are useful to detect hypermethylation at one or more of these genetic loci. In some embodiments, methylation of the vimentin locus may be assayed.

In one embodiment, scanning for one or more mutations at the APC-MCR may detect adenomas with greater than 74% sensitivity.

In one embodiment, the following panel may be used to detect adenomas with greater than 90% sensitivity: scanning for one or more mutations in the APC-MCR locus, exon 9 of the PIK3CA locus, exon 20 of the PIK3CA locus, B-catenin (e.g., exon 5), or a mutation in BRAF that results in a V599E amino acid change. Scanning as described herein can be used to detect one or more mutations in the APC-MCR locus, exon 9 of the PIK3CA locus, or exon 20 of the PIK3CA locus. Mutations at the BRAF locus can be detected via primer extension or other appropriate methodology.

In one embodiment, a combination of all of the above loci may be used to detect adenomas with a greater than 95% sensitivity (e.g., greater than 98% sensitivity).

Appropriate capture probes may be used to capture target nucleic acid molecules that contain one or more of the above regions of interest. Similarly, appropriate analytical or sequencing primers (e.g., primers between about 10 and about 40, or about 15 and about 30 bases long) may be used to interrogate these regions for the presence of a mutant or altered nucleotide associated with an adenoma.

Similarly, other combinations of one or more of these and/or other genomic region(s) associated with adenomas, early stage cancer or other diseases may be captured and interrogated for the presence of these or other known mutations or alterations associated with adenoma and or cancer (e.g., colorectal adenoma or cancer).

### EXAMPLES

### Example 1

### Stool Sample Preparation

Sample Collection and Recovery of DNA from Stool

Stool samples may be frozen within 1 hour of defecation, and shipped on dry ice (−78° C.) for processing and analysis. Once received, samples may be subjected to different stabilization and/or processing techniques.

Different sample preparation methodologies used to recover DNA from stool have been previously reported (Ahlquist D A, Skoletsky J E, Boynton K A, et al. Colorectal cancer screening by detection of altered human DNA in stool: Feasibility of a multi-target assay panel. Gastroenterology 2000; 119:1219-1227; Whitney D, Skoletsky J, Moore K, et al. Enhanced Retrieval of DNA from Human Fecal Samples Results in Improved Performance of Colorectal Cancer Screening Assay. J. Mol. Diagn. 2004; 6 (4), 386-395). Stool aliquots may be weighed and combined with a stabilization buffer (e.g., 0.5M Tris, 0.15M EDTA, and 10 mM NaCl). Stabilization buffer may be added at a ratio of 7:1 volume to stool weight (mls/g). (1:7 (w/v) ratio), and the sample may be homogenized (e.g., on an Exactor (Exact Sciences)). After homogenization, a 4-g stool equivalent (−32 mls) or other amount (e.g., a 30-g equivalent) of sample may be centrifuged to remove all particulate matter. The supernatants may be treated with 20 μl TE buffer (0.01 mol/L Tris [pH 7.4] and 0.001 mol/L EDTA) containing RNase A (2.5 mg/mL), and incubated at 37° C. for 1 hour. Total nucleic acid may then be precipitated (first adding 1/10 volume 3 mol/L NaAc, then an equal-volume of isopropanol). Genomic DNA may be pelleted by centrifugation, the supernatant removed, and the DNA resuspended in TE. A hybrid capture technique may then be used to isolate target DNA molecules of interest.

### Example 2

### Colonic Effluent Processing

Colonic effluent may be processed for example as follows. DNA may be precipitated and resuspended in 7×TNE (EXACT Sciences, Maynard Mass.). The DNA-TNE solution may be centrifuged to pellet residual particulate matter, and the supernatant may be removed and incubated at 37° C. for 30 to 60 minutes in RNAse (4 mg/ml; Sigma Chemical Co., St. Louis, Mo.). The DNA is precipitated in 1/10 volume 3 mol/L sodium acetate (Fisher Scientific, Pittsburgh, Pa.) and an equal volume of isopropyl-alcohol (EM Science, Gibbstown, N.J.), and centrifuged and washed in 70% ethanol. After a final centrifugation, the pellet is air-dried and resuspended in 1×TE. The DNA solution is incubated at room temperature overnight and stored at −20° C.

### Example 3

### Human DNA Purification

Target human DNA fragments may be purified from total nucleic acid preparations using a DNA affinity electrophoresis purification methodology. In brief, human DNA can be separated from the excess bacterial DNA by hybridization of the target sequences to complementary, covalently-bound oligonucleotide capture probes in acrylamide gels membranes. Crude human DNA preparations (240011) may be mixed with 960 μl formamide (Sigma), 385 μl 10×TBE, and filtered through a 0.8 μm syringe filter (Nalgene, Rochester, N.Y.), then denatured (heated at 95 C for 10 min., then cooled in ice for 5 min.). The sample mix may be loaded on top of a capture membrane, and electrodes above and below the capture layer may be applied. Samples may be electrophoresed (15V, 16 h) using TBE in the reservoirs above and below the capture layer. After electrophoretic capture the remaining solution may be removed from the tubes, and the tube array may be separated from the capture plate. The capture membranes then may be washed and 40 μl of 100 mM NaOH may be added to the top of the capture membrane and incubated for 15 min. The capture plate may be placed on top of a custom molded 48-well DNA collection plate and centrifuged briefly (1900×g) to recover the eluted DNA. Then 8 μl of neutralization buffer (500 mM HCL+0.1×TE) may be added to each well of the collection plate and mixed.

In other embodiments, repetitive exposure of a nucleic acid sample (e.g., using reversed-field electrophoresis) may be used as described herein.

Quantification of Recovered Human DNA by Taqman Analysis

TaqMan analysis may be performed on an I-Cycler (BioRad) with primers against a 200-bp region of the APC gene. A probe labeled with 6-carboxyfluorescein (FAM) and 6-carboxytetramethylrhodamine (TAMRA) may be used to detect PCR product. Amplification reactions may consist of captured human stool DNA mixed with 10×PCR buffer, LATaq enzyme (Takara), 1×PCR primers (5 μM), and 1× TaqMan probe (2 μM; Biosearch Technologies). A 5 μl volume of captured DNA may be used in the PCR reactions. TaqMan reactions may be performed using standard conditions or conditions described herein or in the art.

Sequence-Specific Amplification

Polymerase chain reaction (PCR) amplifications (50 μL) may be performed on MJ Research Tetrad Cyclers (Watertown, Mass.) using 10 μL of purified DNA, 10×PCR buffer (Takara Bio Inc; Madison, Wis.), 0.2 mmol/L dNTPs (Promega, Madison, Wis.), 0.5 μmol/L sequence-specific primers (Midland Certified Reagent Co., Midland, Tex.), and 2.5 U LATaq DNA polymerase (Takara). A plurality of amplification reactions may be performed under identical thermocycler conditions: e.g., 94° C. for 5 minutes, and 40 cycles consisting of 94° C. (1 min.), 60° C. (1 min.), and 72° C. (1 min.), with a final extension of 5 minutes at 72° C. For analysis of each of the PCR products, a volume of each amplification reaction may be loaded and electrophoresed on a 4% ethidium bromide-stained NuSieve 3:1 agarose gel (FMC, Rockland, Me.) and visualized with a Stratagene EagleEye II (Stratagene, La Jolla, Calif.) still image system.

A multi-target assay designed to have 13 separate PCR reactions in the multiple mutation (MuMu) panel, and 16 PCR reactions in the DIA portion of the assay may be used. However, other multi-target assays interrogating two or more (e.g., 3, 4, 5, 6, 7, 8, 9, 10, 11, 12, 13, 14, 15, 16, 17, 18, 19, 20, 20-25, 25-30, 30-40, 40-50, or more) different target regions and or different specific mutations or types of mutation may be used according to methods described throughout the specification, including the examples.

Mutation Panel Analysis

The presence or absence of point mutations or Bat-26-associated deletions may be determined by using modified solid-phase single-base extension (SBE) reactions. Point mutation targets may include: codons K12p1, K12p2, and K13p2 on the K-ras gene; codons 876, 1306, 1309, 1312, 1367p1, 1378p1, 1379, 1450p1, 1465 and 1554 on the APC gene; and codons 175p2, 245p1, 245p2, 248p1, 248p2, 273p1, 2'73p2, and 282p1 on the p53 gene. Including the Bat-26 deletion marker, a panel may consist of 22 markers in total. For all gene targets, separate wild-type and mutant specific reactions may be performed. Details of these reactions and analysis using capillary electrophoresis have been previously described (Whitney D, Skoletsky J, Moore K, et al. Enhanced Retrieval of DNA from Human Fecal Samples Results in Improved Performance of Colorectal Cancer Screening Assay. J. Mol. Diagn. 2004; 6 (4), 386-395). Other combinations of these and/or other genetic loci and/or mutations may be interrogated.

### Example 4

### Electrophoretic Media

Electrophoretic media useful in the invention include any media through which charged molecules can migrate in solution in response to an electric field and to which binding partners can be immobilized, including polymeric matrices of gels, packed volumes of particles or beads, and hybrid media including beads or particles embedded in a polymeric gel matrix.

In some embodiments, one or more regions of the electrophoretic medium can be formed from different materials than the other regions (e.g., different polymeric matrices, different packed beads, hybrid gel-bead media, and combinations thereof). The materials for the different regions can be selected according to principles well known in the art to effect different separations or to selectively retain target or non-target molecules.

Polymeric Gel Media

In some embodiments, one or more of the regions of the electrophoretic medium are formed as a polymeric gel. Commonly used gel media useful in the invention include polymeric gels formed from monomers of acrylamide, agarose, starches, dextrans, and celluloses, as well as chemically modified or functionalized variants of these monomers (see, e.g., Polysciences, Inc., Polymer & Monomer catalog, 1996-1997, Warrington, Pa.), (Smithies (1959), Biochem. J. 71:585; Quesada (1997), Curr. Opin. Biotech. 8:82-93).

For the separation of proteins, 5-15% (w/v) polyacrylamide gels are typically used. For small nucleic acid molecules (e.g., <1 kb), 5%-20% (w/v) polyacrylamide gels can be used. For the separation of very large nucleic acid fragments, however, the pore size of standard polyacrylamide gels can be insufficient to allow adequate movement and separation of the fragments. Therefore, lower percentage polyacrylamide gels (e.g., 2-5% (w/v)) can be used. These low percentage polyacrylamide gels, however, have poor mechanical strength. Alternatively, agarose electrophoretic media can be used for nucleic acid gels. For example, gels of 0.5-2.0% (w/v) agarose can be for most nucleic acid separations, and 0.5-1.0% (w/v) gels can be used for larger nucleic acid fragments. Low percentage agarose gels have greater mechanical strength than low percentage polyacrylamide gels.

For some methods, composite gel media containing a mixture of two or more supporting materials can be used. For example, and without limitation, composite acrylamide-agarose gels can be employed which contain from 2-5% (w/v) acrylamide and 0.5%-1.0% (wfv) agarose. In such gels, the polyacrylamide matrix performs provides the major sieving function, whereas the agarose provides mechanical strength for convenient handling without significantly altering the sieving properties of the acrylamide. In composite gels, the binding partners optionally can be attached to the component that performs the major sieving function of the gel, because that component more intimately contacts the target molecules.

In other embodiments, macroporous gels can be formed by mixing the gel-forming materials with organic liquids or pore-forming agents prior to polymerization. These liquids or pore-forming agents can be removed subsequent to polymerization to create a polymeric gel matrix with larger pores. The larger pores are useful for permitting the movement of large target molecules (e.g., genomic fragments) through the polymeric matrix material, while also maintaining the mechanical strength of the medium.

Packed Bead Media

In other embodiments, as an alternative to polymeric gel media, packed volumes of small beads or particle beds can be used as electrophoretic media. Such particle beds, which are frequently used in chromatography, have the advantage of large interstitial voids which allow for the passage of large molecules such as nucleic acid fragments>1 kb. In some embodiments, the beads have average diameters in the range of 1-5 μm, 5-50 μm, or 50-150 μm, although larger beads can also be used. Beads useful in the invention can be formed from materials including, but not limited to, agarose polymers, dextran polymers, acrylic polymers, glass, latex, polystyrene, poly(hydroxyethylcellulose), poly(ethylenoxide), a modified acrylamide, and acrylate ester.

Beads useful in the invention can be solid beads or porous beads, In some embodiments, porous beads will have diameters in the range of 10-20 μm or, more generally 10-50 μm, and can have a wide range of pore sizes. Such porous beads can include binding partners embedded within the pores and/or bound to the surfaces of the probes. Non-porous or solid beads can have a wider range of diameters, including without limitation beads in the range of 1-100 μm.

Such beads conveniently can be coated (including the interiors of pores) with one member of an affinity binding pair such that binding partners bound to the other member of the affinity binding pair can be immobilized on the beads. For example, and without limitation, beads can be coated with avidin or streptavidin and binding partners can be conjugated to biotin to cause immobilization of the binding partners on the beads. Similarly, probes can be coated with Protein A to immobilize antibody binding partners that bind to Protein A.

Beads also can be treated or coated to reduce non-specific binding or target or other molecules in a sample. For example, beads can be treated to reduce the number of hydrophobic groups (e.g., benzyl groups) on the surface, or to increase the number of hydrophilic groups (e.g., carboxyl groups) on the surface. Beads can also be coated with gelatin, bovine serum albumin or other molecules that will non-specifically bind to and “block” the surface prior to use with test samples.

In embodiments employing beads as electrophoretic media, it may be necessary to separate different regions of the electrophoretic medium by separators which are membranes or meshes that prevent the movement of the beads from one region to another in response to the electric field. Such separators must have pores sufficiently large to be permeable to the target molecules, but not permeable to the beads. Such separators can be used alone, or in combination with spacer elements or other structures between regions of the electrophoretic medium.

Hybrid Gel-Bead Media

In other embodiments, hybrid media can be formed which include small beads or particles embedded or enmeshed in a polymeric gel. Such hybrid-gel media can be formed from any of the polymeric gel materials and any of the bead materials described above. For example, and without limitation, polyacrylate or polystyrene beads can be embedded in a polyacrylamide or agarose gel matrix. In some embodiments, the binding partners will be bound to the beads prior to production of the hybrid gel-bead media. In other embodiments, however, the binding partners can be co-polymerized into the polymeric gel during its formation, or can be bound to the hybrid gel-bead media after formation.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. Certain advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

What is claimed is:

1. A clinical algorithm for selecting a patient for invasive testing, the method comprising
performing a virtual colonoscopy on a patient:
collecting a colonic effluent sample from the patient;
performing a molecular assay on the colonic effluent; and
performing an invasive analysis on the patient if the patient is identified as positive for a disease in both the virtual colonoscopy and the molecular assay.

2. The method of claim 1, wherein the patient is human.

3. The method of claim 1, wherein the molecular assay is performed on the colonic effluent only if the patient is identified as positive in the virtual colonoscopy.

4. A method for excluding patients from undergoing an invasive colonic testing, the method comprising:
performing a virtual colonoscopy on a population of subjects;
collecting a colonic effluent from each subject in the population;
identifying candidates for invasive colonic testing as those subjects that are positive for indicia of colonic disease in the virtual colonoscopy; and,
excluding candidates from the invasive colonic testing if they are negative in a molecular assay performed on their colonic effluent.

5. A patient care algorithm comprising:
performing a virtual colonoscopy on a subject;
performing a molecular analysis on a colonic sample obtained from the subject if the virtual colonoscopy is positive
performing an invasive colonic test on the subject if the molecular analysis is positive.

6. The method of any one of claims 1-5, wherein the molecular assay is an assay capable of detecting one or more abnormal nucleic acids associated with a disease.

7. The method of claim 6, wherein the disease is colon cancer.
